(19) United States
(12) Patent Application Publication
(10) Pub. No.: US 2024/0260577 A1
(43) Pub. Date: Aug. 8, 2024

(54) INSECT, ACARINA AND NEMATODE PEST CONTROL

(71) Applicant: SYNGENTA CROP PROTECTION AG, Basel (CH)

(72) Inventors: Patrik HOEGGER, Stein (CH); Ottmar Franz HUETER, Stein (CH); Remo WINTER, Stein (CH)

(73) Assignee: SYNGENTA CROP PROTECTION AG, Basel (CH)

(21) Appl. No.: 18/560,899

(22) PCT Filed: May 13, 2022
(86) PCT No.: PCT/EP2022/063096

§ 371 Date: Nov. 14, 2023

(30) Foreign Application Priority Data
May 14, 2021 (EP) 21173976.8
Jul. 7, 2021 (EP) 21184391.7

(51) Int. Cl. A01N 43/56; A01P 3/00; A01P 5/00; A01P 7/02; A01P 7/04; A61K 31/4439; A61K 45/06; C07D 401/04

(52) U.S. Cl. CPC A01N 43/56; A01P 3/00; A01P 5/00; A01P 7/02; A01P 7/04; A61K 31/4439; A61K 45/06; C07D 401/04

(57) ABSTRACT

The present invention relates to combinations of a compound of formula (I), wherein: R¹ is hydrogen or fluorine; and R² is: in which n is 0, 1 or 2; with a compound selected from a group consisting of mixing partners; and to methods of controlling or preventing damage to plants and infestations of animals; and to formulations, concentrates and seed treatments comprising such combinations.

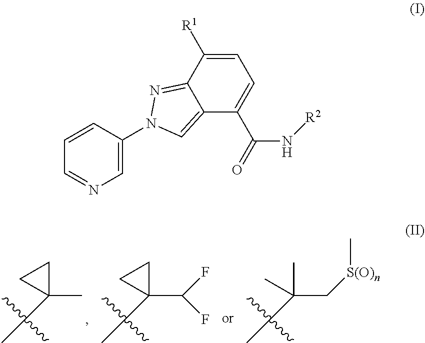

The present invention relates to a combination for the control of damage caused by insect, acarina and/or nematode pests, in particular on plants and animals, which combination comprises a mixture of active ingredients.

Certain active ingredients and combinations of active ingredients for controlling pest attack are described in the literature. For example, WO2020/018362A1 and WO2021/007545A1 disclose substituted indazole compounds for use in controlling invertebrate pests. There is a continuing need to provide pesticidal combinations, which provide improved, for example, biological properties, for example, synergistic properties, especially for controlling insect, acarina and nematode pests. The benefits may also be an increased safety profile, improved physico-chemical properties, or increased biodegradability.

It is now been found that particular combinations of active ingredients provide unexpected control or prevention of damage to a plant or animal, when the particular combination is applied on the plant, the locus thereof or its propagation material or to the animal.

Accordingly, in a first aspect, the present invention provides a combination or composition comprising, as component (A), a compound of formula (I):

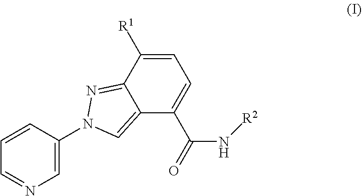

wherein:
R¹ is hydrogen or fluorine; and
R² is:

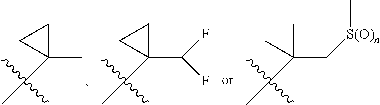

wherein n is 0, 1 or 2;
or a salt thereof;
and, as component (B), one or more of a compound selected from the group consisting of:
   B-1 (S)-N-[[2-fluoro-4-[2-hydroxy-3-(3,4,5-trichlorophenyl)-3-(trifluoromethyl)pyrrolidin-1-yl]phenyl]methyl]cyclopropanecarboxamide (CAS 2044701-44-0)
   B-2 N-[[2-fluoro-4-[2-hydroxy-3-(3,4,5-trichlorophenyl)-3-(trifluoromethyl)pyrrolidin-1-yl]phenyl]methyl]cyclopropanecarboxamide (CAS 1365070-72-9)
   B-3 spidoxamat (CAS 907187-07-9)
   B-4 fenmezoditiaz (CAS 2413390-32-4)
   B-5 3-(2-chlorothiazol-5-yl)-8-methyl-5-oxo-6-phenyl-2,3-dihydrothiazolo[3,2-a]pyrimidin-8-ium-7-olate (CAS 2413839-57-1)
   B-6 (3R)-3-(2-chlorothiazol-5-yl)-8-methyl-5-oxo-6-phenyl-2,3-dihydrothiazolo[3,2-a]pyrimidin-8-ium-7-olate, enolate tautomer (CAS 2413839-58-2)
   B-7 (3R)-3-(2-chlorothiazol-5-yl)-8-methyl-5-oxo-6-phenyl-2,3-dihydrothiazolo[3,2-a]pyrimidin-8-ium-7-olate, diketone (CAS 2246757-58-2)
   B-8 1-[6-(2,2-difluoro-7-methyl-[1,3]dioxolo[4,5-f]benzimidazol-6-yl)-5-ethylsulfonyl-3-pyridyl]cyclopropanecarbonitrile (CAS 2408220-94-8)
   B-9 2-[3-ethylsulfonyl-5-(trifluoromethyl)pyrazolo[1,5-a]pyridin-2-yl]-3-methyl-6-(trifluoromethyl)imidazo[4,5-b]pyridine (CAS 2133042-44-9)
   B-10 2-[3-ethylsulfonyl-6-(trifluoromethyl)pyrazolo[1,5-a]pyridin-2-yl]-3-methyl-6-(trifluoromethyl)imidazo[4,5-b]pyridine (CAS 2133042-31-4)
   B-11 (3-ethylsulfonyl-2-pyridyl)-5-(2,2,3,3,3-pentafluoropropoxy)pyrazine (CAS 2095470-94-1) B-12 2-[3-ethylsulfonyl-6-(1,2,4-triazol-1-yl)-2-pyridyl]-3-methyl-6-(trifluoromethyl)imidazo[4,5-b]pyridine (CAS 1808115-49-2)
   B-13 carbosulfan (CAS 55285-14-8)
   B-14 1-(2-cyanoethyl)-4-oxo-3-[3-(trifluoromethyl)phenyl]pyrido[1,2-a]pyrimidin-1-ium-2-olate (CAS 2032403-97-5)
   B-15 2-(5-cyclopropyl-3-ethylsulfonyl-2-pyridyl)-5-(trifluoromethylsulfonyl)-1,3-benzoxazole (CAS 2128706-05-6)
   B-16 6-(5-cyclopropyl-3-ethylsulfonyl-2-pyridyl)-2,2-difluoro-7-methyl-[1,3]dioxolo[4,5-f]benzimidazole (CAS 2408220-91-5)
   B-17 azocyclotin (CAS 41083-11-8)
   B-18 pyrimidifen (CAS 105779-78-0)
   B-19 fluacrypyrim (CAS 229977-93-9)
   B-20 diflovidazin (CAS 162320-67-4)
   B-21 (R)-(2Z)-2-[2-fluoro-4-methyl-5-(2,2,2-trifluoroethylsulfinyl)phenyl]imino-3-(2,2,2-trifluoroethyl)thiazolidin-4-one (CAS 2377084-09-6)
   B-22 2-[2-fluoro-4-methyl-5-(2,2,2-trifluoroethylsulfinyl)phenyl]imino-3-(2,2,2-trifluoroethyl)thiazolidin-4-one (CAS 1445684-82-1)
   B-23 (2Z)-2-[2-fluoro-4-methyl-5-(2,2,2-trifluoroethylsulfinyl)phenyl]imino-3-(2,2,2-trifluoroethyl)thiazolidin-4-one (CAS 1445683-71-5)
   B-24 4-dimethyl-2-[2-(3-pyridyl)indazol-5-yl]-1,2,4-triazolidine-3,5-dione (CAS 2171099-09-3)
   B-25 N-[3-chloro-1-(3-pyridyl)pyrazol-4-yl]-2-methylsulfonyl-propanamide (CAS 2396747-83-2)
   B-26 lepimectin (CAS 863549-51-3)
   B-27 milbemectin (CAS 1799297-76-9 and CAS 51596-10-2)
   B-28 cyproflanilide (CAS 2375110-88-4)
   B-29 thiocyclam (CAS 31895-21-3)
   B-30 guadipyr (CAS 1227849-60-6)
   B-31 5-[[(1R,3R)-3-[3,5-bis(trifluoromethyl)phenyl]-2,2-dichloro-cyclopropanecarbonyl]amino]-2-chloro-N-[3-[(2,2-difluoroacetyl)amino]-2,4-difluoro-phenyl]benzamide) (CAS 2220132-55-6)
   B-32 5-[[trans-3-[3,5-bis(trifluoromethyl)phenyl]-2,2-dichloro-cyclopropanecarbonyl]amino]-2-chloro-N-[3-[(2,2-difluoroacetyl)amino]-2,4-difluoro-phenyl]benzamide
   B-33 afoxolaner (CAS 1093861-60-9)
   B-34 fluralaner (CAS 864731-61-3)
   B-35 sarolaner (CAS 1398609-39-6)
   B-36 ivermectin (CAS 70288-86-7)
   B-37 tigolaner (CAS 1621436-41-6)
   B-72 oxathiapiprolin
   B-73 iprodione
   B-74 fludioxonil
   B-75 mefenoxam or metalaxyl-M
   B-76 metalaxyl
   B-77 penflufen
   B-78 fluindapyr
   B-79 penthiopyrad
   B-80 furametpyr
   B-81 pydiflumetofen
   B-82 sedaxane
   B-83 thiabendazole
   B-84 mefentrifluconazole
   B-85 prothioconazole
   B-86 triticonazole
   B-87 flutriafol
   B-88 difenconazole
   B-89 ipconazole
   B-90 azoxystrobin
   B-91 fluoxastrobin
   B-92 picarbutrazox
   B-93 amisulbrom
   B-94 cyazofamid
   B-95 hymexazole
   B-96 fluopicolide
   B-97 fluoxapiprolin
   B-98 cyprodinil
   B-99 thiram
   B-100 florylpicoxamid
   B-101 fenamidone
   B-102 famoxadone
   B-103 oxadixyl
   B-104 acibenzolar-S-methyl
   B-105 boscalid
   B-106 bixafen
   B-107 fluopyram
   B-108 fluxapyroxad
   B-109 isoflucypram
   B-110 flutolanil
   B-111 pyraziflumid
   B-112 pyrapropoyne
   B-113 inpyrfluxam
   B-114 carboxin
   B-115 zoxamide
   B-116 thiophanate-methyl
   B-117 ethaboxam
   B-118 dimethomorph
   B-119 metconazole
   B-120 prochloraz
   B-121 tebuconazole
   B-122 triadimenol
   B-123 fluquinconazole
   B-124 myclobutanil
   B-125 imazalil
   B-126 oxpoconazole
   B-127 cyproconazole
   B-128 pyraclostrobin
   B-129 picoxystrobin
   B-130 enestrobin
   B-131 fenaminostrobin
   B-132 mandestrobin
   B-133 metyltetraprole
   B-134 fluazinam
   B-135 quinofumelin
   B-136 cyflufenamid (CAS 180409-60-3)
   B-137 ipflufenoquin
   B-138 cymoxanil
   and
   B-139 copper
or salts thereof.

In a second aspect, the present invention provides a method of controlling or preventing damage to a plant, which comprises applying, in any desired sequence or simultaneously, on the plant, the locus thereof or its propagation material, a combination comprising, as component (A), a compound of formula (I) as defined above or an agrochemically acceptable salt thereof; and, as component (B), one or more compounds selected from the group consisting of B-1 to B-37 as defined above and/or one or more compounds selected from the group consisting of B-72 to B-139 as defined above, or agrochemically acceptable salts thereof.

In some examples of the first and second aspects of the present invention, component (B) is a compound selected from the group consisting of compounds B-1 to B-37 defined above.

In other examples of the first and second aspects of the present invention, component (B) is a compound selected from the group consisting of compounds B-72 to B-139 as defined above.

In yet further examples of the first and second aspects of the present invention, component (B) further comprises a compound selected from B-38 afidopyropen (CAS 915972-17-7)
B-39 flonicamid (CAS 158062-67-0)
B-40 fluxametamide (CAS 928783-29-3)
B-41 spirotetramat (CAS 203313-25-1)
B-42 triflumezopyrim (CAS 1263133-33-0)
B-43 broflanilide (CAS 1207727-04-5)
B-44 nicofluprole (CAS 1771741-86-6)
B-45 spinetoram (CAS 935545-74-7)
B-46 thiacloprid (CAS 111988-49-9)
B-47 flupyradifurone (CAS 951659-40-8)
B-48 imidacloprid (CAS 138261-41-3)
B-49 sulfoxaflor (CAS 946578-00-3)
B-50 acetamiprid (CAS 135410-20-7)
B-51 nitenpyram (CAS 150824-47-8)
B-52 clothianidin (CAS 210880-92-5)
B-53 thiamethoxam (CAS 153719-23-4)
B-54 tetraniliprole (CAS 1229654-66-3)
B-55 flubendiamide (CAS 272451-65-7)
B-56 chlorantraniliprole (CAS 500008-45-7)
B-57 cyantraniliprole (CAS 736994-63-1)
B-58 oxazosulfyl (CAS 1403615-77-9)
B-59 indoxacarb (CAS 173584-44-6)
B-60 cyfluthrin (CAS 68359-37-5)
B-61 deltamethrin (CAS 52918-63-5)
B-62 acrinathrin (CAS 101007-06-1)
B-63 zeta-cypermethrin (CAS 1315501-18-8)
B-64 bifenthrin (CAS 82657-04-3)
B-65 gamma-cyhalothrin (CAS 76703-62-3)
B-66 alpha-cypermethrin (CAS 67375-30-8)
B-67 permethrin (CAS 52645-53-1)
B-68 fenpropathrin (CAS 39515-41-8)
B-69 fenvalerate (CAS 51630-58-1)
B-70 esfenvalerate (CAS 66230-04-4)
B-71 lambda-cyhalothrin (CAS 91465-08-6)
B-140 isocycloseram (CAS 2061933-85-3)
B-141 tefluthrin (CAS 79538-32-2)
B-142 cyclobutrifluram (CAS 1644251-74-0)
B-143 dimpropyridaz (CAS 1403615-77-9)
and
B-144 fipronil (CAS 120068-37-3)
   or agrochemically acceptable salts thereof.

In a third aspect, the present invention provides a method of controlling or preventing infestation of an animal, which comprises applying, in any desired sequence or simultaneously, on the animal a combination comprising, as component (A), a compound of formula (I) as defined above or a pharmaceutically or veterinary acceptable salt thereof; and, as component (B), a compound selected from the group consisting of B-33 to B-37 as defined above, or pharmaceutically or veterinary acceptable salts thereof.

In a fourth aspect, the present invention provides the use of a combination or composition comprising, as component (A), a compound of formula (I) as defined above or a pharmaceutically or veterinary acceptable salt thereof; and, as component (B), a compound selected from the group consisting of B-33 to B-37 as defined above, or pharmaceutically or veterinary acceptable salts thereof, in controlling or preventing infestation of an animal.

Optionally, the composition further comprises one or more auxiliaries and/or diluents.

The combination or composition of the compound of formula (I) of component (A) and the or each compound of component (B) may be in a suitable ratio by weight, examples of which are between 500:1 to 1:500, 100:1 to 1:100, 50:1 to 1:50, or 20:1 to 1:20, or preferably 10:1 to 1:10 or 5:1 to 1:5; more preferably 3.1 to 1:3 by weight. In certain examples, the ratio is within the range of 2:1 to 1:2, advantageously 1:1, by weight.

In a fifth aspect, the present invention provides a method of controlling or preventing damage to a plant, which comprises applying, in any desired sequence or simultaneously, on the plant, the locus thereof or its propagation material, a combination comprising, as component (A), a compound of formula (I):

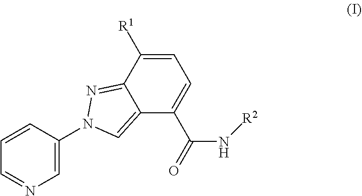

wherein:
R¹ is hydrogen or fluorine; and
R² is:

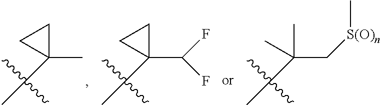

wherein n is 0, 1 or 2;
or an agrochemically acceptable salt thereof;
and, as component (B), one or more of a compound selected from the group consisting of:
   B-38 afidopyropen (CAS 915972-17-7)
   B-39 flonicamid (CAS 158062-67-0)
   B-40 fluxametamide (CAS 928783-29-3)
   B-41 spirotetramat (CAS 203313-25-1)
   B-42 triflumezopyrim (CAS 1263133-33-0)
   B-43 broflanilide (CAS 1207727-04-5)
   B-44 nicofluprole (CAS 1771741-86-6)
   B-45 spinetoram (CAS 935545-74-7)
   B-46 thiacloprid (CAS 111988-49-9)
   B-47 flupyradifurone (CAS 951659-40-8)
   B-48 imidacloprid (CAS 138261-41-3)
   B-49 sulfoxaflor (CAS 946578-00-3)
   B-50 acetamiprid (CAS 135410-20-7)
   B-51 nitenpyram (CAS 150824-47-8)
   B-52 clothianidin (CAS 210880-92-5)
   B-53 thiamethoxam (CAS 153719-23-4)
   B-54 tetraniliprole (CAS 1229654-66-3)
   B-55 flubendiamide (CAS 272451-65-7)
   B-56 chlorantraniliprole (CAS 500008-45-7)
   B-57 cyantraniliprole (CAS 736994-63-1)
   B-58 oxazosulfyl (CAS 1403615-77-9)
   B-59 indoxacarb (CAS 173584-44-6)
   B-60 cyfluthrin (CAS 68359-37-5)
   B-61 deltamethrin (CAS 52918-63-5)
   B-62 acrinathrin (CAS 101007-06-1)
   B-63 zeta-cypermethrin (CAS 1315501-18-8)
   B-64 bifenthrin (CAS 82657-04-3)
   B-65 gamma-cyhalothrin (CAS 76703-62-3)
   B-66 alpha-cypermethrin (CAS 67375-30-8)
   B-67 permethrin (CAS 52645-53-1)
   B-68 fenpropathrin (CAS 39515-41-8)
   B-69 fenvalerate (CAS 51630-58-1)
   B-70 esfenvalerate (CAS 66230-04-4)
   B-71 lambda-cyhalothrin (CAS 91465-08-6)
   B-140 isocycloseram
   B-141 tefluthrin
   B-142 cyclobutrifluram
   B-143 dimpropyridaz
   and
   B-144 fipronil
or agrochemically acceptable salts thereof;
wherein the combination of the compound of formula (I) of component (A) and the or each compound of component (B) is in a ratio by weight of 500:1 to 1:500, 100:1 to 1:100, 50:1 to 1:50, or 20:1 to 1:20, 10:1 to 1:10; 5:1 to 1:5; or 3.1 to 1:3 by weight.

In a sixth aspect, the present invention provides a combination or composition comprising, as component (A), a compound of formula (I):

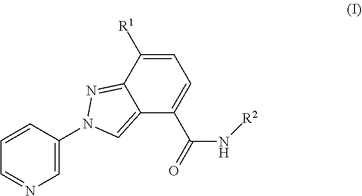

wherein:
R¹ is hydrogen or fluorine; and
R² is:

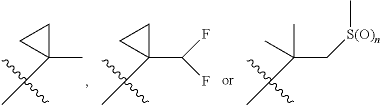

wherein n is 0, 1 or 2;
or an agrochemically acceptable salt thereof;
and, as component (B), a compound selected from the group consisting of B-38 to B-71 and B-140 to B-144 as defined above, or agrochemically acceptable salts thereof;
wherein the composition comprises (A) and (B) in a ratio by weight of 500:1 to 1:500, 100:1 to 1:100, 50:1 to 1:50, or 20:1 to 1:20, 10:1 to 1:10; 5:1 to 1:5; or 3.1 to 1:3 by weight.

Optionally, the composition further comprises one or more auxiliaries and/or diluents.

In certain examples, the ratio of component (A) to the or each compound of component (B) is within the range of 3:1 to 1:3, 2:1 to 1:2, or about 1:1, by weight.

In some examples of the aspects of the present invention, component (B) is a compound selected from compounds B-38, B-40, B-42, B44, B-46, B-51, B-53, B-55, B-60, B-61, B-62, B-63, B-66, B-68, B-69, B-70, B-71, B-141 and B-142.

In other examples of the aspects of the present invention, component (B) is selected from compounds B-43, B-52, B-53, B-58, B-65, B-140 and B-143.

In further examples of the aspects of the present invention, component (B) is selected from compounds B-39, B-41, B-45, B-47, B-48, B-49, B-50, B-56, B-57, B-58, B-64, B-67, and B-144.

In some examples of the present invention, component (B) is selected from compounds B-72 to B-91, B-93, B-94, B-95 to B-125, B-127, B-128, B-129 and B-132 to B-138.

In a seventh aspect, the present invention provides the use of a composition of the invention as an insecticide, acaricide and/or nematicide.

In an eighth aspect, the present invention provides a formulation comprising a composition of the invention, the formulation comprising from 0.01 to 90% by weight of active agents, from 0 to 25% by weight of agriculturally or pharmaceutically acceptable surfactant and from 10 to 99.9 percent by weight of solid or liquid formulation inerts and adjuvants.

In a ninth aspect, the present invention provides a concentrate composition for dilution by a user, the concentrate composition comprising a composition of the invention, comprising from 2% to 80% by weight, preferably between about 5% and 70% by weight of active agents.

In a tenth aspect, the present invention also provides the use of the combination or composition as defined above for controlling insect, acarina and/or nematode pests.

In certain examples, the compound of formula (I) is a compound of formula (I) in which R¹ is hydrogen and R² is

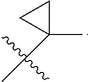 [SMILES: *C1(C)CC1 ; "."]

In certain examples, the compound of formula (I) is a compound of formula (I) in which R¹ is hydrogen and R² is

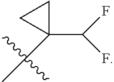 [SMILES: *C1(C(F)F)CC1]

In certain examples, the compound of formula (I) is a compound of formula (I) in which R¹ is fluorine and R² is

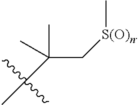

wherein n is 0, 1 or 2.

In preferred embodiments of the various aspects of the present invention, the compound of formula (I) is compound of formula (A-1)

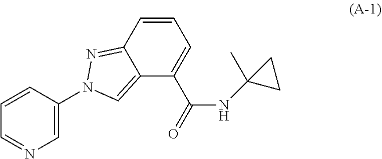 [SMILES: CC1(NC(=O)c2cccc3nn(-c4cccnc4)cc23)CC1]

Besides any synergistic action, the combinations according to the first, second, fourth and fifth aspects of the invention can also have further surprising advantageous properties. Examples of such advantageous properties that may be mentioned are: more advantageous degradability, improved toxicological and/or ecotoxicological behaviour, or improved characteristics of the useful plants including: emergence, crop yields, more developed root system, tillering increase, increase in plant height, bigger leaf blade, less dead basal leaves, stronger tillers, greener leaf colour, less fertilizers needed, less seeds needed, more productive tillers, earlier flowering, early grain maturity, less plant verse (lodging), increased shoot growth, improved plant vigour, and early germination.

Certain weight ratios of component (A) to component (B) may give rise to synergistic activity. Therefore, according to a further aspect of the invention there is provided a composition, wherein component (A) and component (B) are present in the composition in amounts producing a synergistic effect. This synergistic activity is apparent from the fact that the activity of the composition comprising component (A) and component (B) is greater than the sum of the corresponding activities of component (A) and of component (B) alone. This synergistic activity extends the range of action of component (A) and component (B) in two ways. Firstly, the rates of application of component (A) and component (B) are lowered whilst the action remains equally good, meaning that the active ingredient mixture still achieves a high degree of pest control even where the two individual components have become totally ineffective in such a low application rate range. Secondly, there is a substantial broadening of the spectrum of pests that can be controlled.

The active ingredients in the combinations of the present invention may be applied to a pest, plant, plant propagation material or plant growing locus either simultaneously (for example as a pre-formulated mixture or a tank mix), or sequentially in a suitable timescale.

The compounds of the combination (i.e. components (A), and (B)), and any other pesticides, may be used either in pure form, i.e., as a solid active ingredient, for example, in a specific particle size, or preferably together with at least one of the auxiliary (also known as adjuvants) customary in formulation technology, such as extenders, e.g., solvents or solid carriers, or surface-active compounds (surfactants), in the form of a formulation, in the present invention. Generally, the compounds (A), and (B) are each in the form of a formulation composition with one or more of customary formulation auxiliaries.

Therefore, compounds (A) and (B) can be used in the form of separate formulations. The compounds can be applied to the locus where control is desired either simultaneously or in succession at short interval, for example on the same day, if desired together with further carriers, surfactants or other application-promoting adjuvants customarily employed in formulation technology. In a preferred embodiment, (A) and (B) are applied simultaneously. Co-application or simultaneous application of components (A) and (B) has the added benefit of minimising farmer time spent applying products to crops. The combination may also encompass specific plant traits incorporated into the plant using any means, for example conventional breeding or genetic modification.

When compounds of the combination (i.e. (A), and (B)) are applied simultaneously in the present invention, they may be applied as a composition containing the combination, in which case each of (A), and (B) can be obtained from a separate formulation source and mixed together (known as a tank-mix, ready-to-apply, spray broth, or slurry), optionally with other pesticides, or (A), and (B) can be obtained as single formulation mixture source (known as a pre-mix, concentrate, formulated product), and optionally mixed together with other pesticides.

In one embodiment, the composition comprises an agriculturally acceptable formulation adjuvant. In a further embodiment, there is provided a composition consisting essentially of component (A), component (B) and an agriculturally acceptable adjuvant. In a further embodiment, there is provided a composition consisting of component (A), component (B) and an agriculturally acceptable adjuvant. The compositions of the present invention are generally formulated using formulation adjuvants, such as carriers, solvents and surface-active agents (SFAs).

The compounds in Table 1 below illustrate certain compounds of formula (I) in the combinations and compositions of the invention.

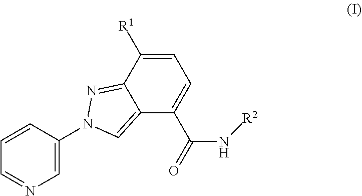

TABLE 1
This table discloses five specific compounds of formula (I):

| Compound Number | R¹ | R² |
|---|---|---|
| A-1 | H | 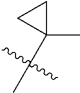 [SMILES: *C1(C)CC1] |
| A-2 | H | 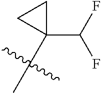 [SMILES: *C1(C(F)F)CC1] |
| A-3 | F | 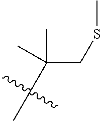 [SMILES: *C(C)(C)CSC] |
| A-4 | F | 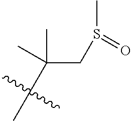 [SMILES: *C(C)(C)CS(C)=O] |
| A-5 | F | 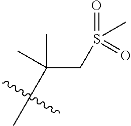 [SMILES: *C(C)(C)CS(C)(=O)=O] |

The combinations and compositions of the present invention may be useful for the control of posts, such as insects, in improving the tolerance of crop plants to abiotic stress conditions, and/or in improving the yield of crop plants. In one embodiment, the combinations and compositions of the present invention may be useful for the control of insect and/or acarina and/or nematode pests. The present invention provides a method for controlling pests in or on crop plants, improving the tolerance of crop plants to abiotic stress conditions, and/or improving the yield of crop plants, comprising treating the pests, plants, plant part, plant propagation material, or plant growing locus with a composition as described herein.

The combinations and compositions of the present invention may be useful for extending the duration of protection afforded to the plant material. In one embodiment, the combinations and compositions of the present invention may show both a fast-acting curative action and a preventative or protective action.

The combinations and compositions of the present invention may be useful for extending the range of crops with which the combinations and compositions are useful and/or the range of pests against which the combinations and compositions provide effective control.

Accordingly, the combinations and compositions of the present invention provide an enhanced biological profile which may include a more complete activity spectrum and/or complementary modes of activity. Advantageously, the component (A) compounds and the component (B) compounds may provide complementary mobilities in the plant. In certain combinations, the component (A) compound has a greater acropetal movement in the plant compared with the component (B) compound, such that the component (B) compound provides more localised protection.

In other combinations, the component (A) compound has a lower acropetal movement in the plant compared with the component (B) compound, such that the component (A) compound provides more localised protection.

Where used herein, the indication ‘CAS’ followed by a sequence of numbers refers to the Chemical Abstracts Registry number of the active ingredient. Where available or known, active ingredients are also referred to by their common name allocated in accordance with ‘ISO 1750:1981—Pesticides and other agrochemicals—Common names’.

Compounds of formula (I) are described in WO 2015/038503.

Compounds B-1 to B-71 and B-140 to B-144 are known to have insecticidal, nematocidal and/or acaricidal activity. Compounds B-72 to B-139 are known to have fungicidal activity.

B-1, (S)-N-[[2-fluoro-4-[2-hydroxy-3-(3), 4),5-trichlorophenyl)-3-(trifluoromethyl)pyrrolidin-1-yl]phenyl]-methyl]cyclopropanecarboxamide (CAS 2044701-44-0), is described in WO 2012035011. B-2, N-[[2-fluoro-4-[2-hydroxy-3-(3), 4), 5-trichlorophenyl)-3-(trifluoromethyl)pyrrolidin-1-yl]phenyl]methyl]cyclopropanecarboxamide (CAS 1365070-72-9), is described in WO 2012035011. B-3, spidoxamat (CAS 907187-07-9), is described in WO 2006089633. B-4, fenmezoditiaz (CAS 2413390-32-4), is described in WO 2018177970. B-5, 3-(2-chlorothiazol-5-yl)-8-methyl-5-oxo-6-phenyl-2),3-dihydrothiazolo[3),2-a]pyrimidin-8-ium-7-olate (CAS 2413839-57-1), is described in WO 2018177970. B-6, (3R)-3-(2-chlorothiazol-5-yl)-8-methyl-5-oxo-6-phenyl-2),3-dihydrothiazolo[3),2-a]pyrimidin-8-ium-7-olate), enolate tautomer (CAS 2413839-58-2), is described in WO 2018177970. B-7, (3R)-3-(2-chlorothiazol-5-yl)-8-methyl-5-oxo-6-phenyl-2),3-dihydrothiazolo[3),2-a]pyrimidin-8-ium-7-olate), diketone (CAS 2246757-58-2), is described in WO 2018177970. B-8, 1-[6-(2),2-difluoro-7-methyl-[1),3]dioxolo[4),5-f]benzimidazol-6-yl)-5-ethylsulfonyl-3-pyridyl]cyclopropanecarbonitrile (CAS 2408220-94-8), is described in WO 2020013147. B-9, 2-[3-ethylsulfonyl-5-(trifluoromethyl)pyrazolo[1), 5-a]pyridin-2-yl]-3-methyl-6-(trifluoromethyl)imidazo[4), 5-b]pyridine (CAS 2133042-44-9), is described in WO 2017155103. B-10, 2-[3-ethylsulfonyl-6-(trifluoromethyl)pyrazolo[1), 5-a]pyridin-2-yl]-3-methyl-6-(trifluoromethyl)imidazo[4),5-b]pyridine (CAS 2133042-31-4), is described in WO 2017155103. B-11, (3-ethylsulfonyl-2-pyridyl)-5-(2),2),3), 3), 3-pentafluoropropoxy)pyrazine (CAS 2095470-94-1), is described in WO 2017065228. B-12, 2-[3-ethylsulfonyl-6-(1), 2), 4-triazol-1-yl)-2-pyridyl]-3-methyl-6-(trifluoromethyl)imidazo[4),5-b]pyridine (CAS 1808115-49-2), is described in WO 2015133603. B-13, carbosulfan (CAS 55285-14-8), is described in DE 2433680 (1975). B-14, 1-(2-cyanoethyl)-4-oxo-3-[3-(trifluoromethyl)phenyl]pyrido[1),2-a]pyrimidin-1-ium-2-olate (CAS 2032403-97-5), is described in WO 2016171053. B-15, 2-(5-cyclopropyl-3-ethylsulfonyl-2-pyridyl)-5-(trifluoromethylsulfonyl)-1), 3-benzoxazole (CAS 2128706-05-6), is described in WO 2017146226. B-16, 6-(5-cyclopropyl-3-ethylsulfonyl-2-pyridyl)-2), 2-difluoro-7-methyl-[1), 3]dioxolo[4), 5-f]benzimidazole (CAS 2408220-91-5), is described in WO 2020013147. B-17, azocyclotin (CAS 41083-11-8), is described in DE 2143252 (1973). B-18, pyrimidifen (CAS 105779-78-0), is described in EP 196524 (1986). B-19, fluacrypyrim (CAS 229977-93-9), is described in WO 9935914. B-20, diflovidazin (CAS 162320-67-4), is described in CA 2128378 (1995). B-21, (R)-(2Z)-2-[2-fluoro-4-methyl-5-(2),2),2-trifluoroethylsulfinyl)phenyl]imino-3-(2),2),2-trifluoroethyl)thiazolidin-4-one (CAS 2377084-09-6), is described in WO 2013092350. B-22, 2-[2-fluoro-4-methyl-5-(2), 2), 2-trifluoroethylsulfinyl)phenyl]imino-3-(2),2), 2-trifluoroethyl)thiazolidin-4-one (CAS 1445684-82-1), is described in WO 2013092350. B-23, (2Z)-2-[2-fluoro-4-methyl-5-(2),2),2-trifluoroethylsulfinyl)phenyl]imino-3-(2),2), 2-trifluoroethyl)thiazolidin-4-one (CAS 1445683-71-5), is described in WO 2013092350. B-24, 4-dimethyl-2-[2-(3-pyridyl)indazol-5-yl]-1),2),4-triazolidine-3),5-dione (CAS 2171099-09-3), is described in WO 2018011111. B-25, N-[3-chloro-1-(3-pyridyl)pyrazol-4-yl]-2-methylsulfonyl-propanamide (CAS 2396747-83-2), is described in WO 2019236274. B-26, lepimectin (CAS 863549-51-3), is described in WO 2020108146. B-27, milbemectin (CAS 1799297-76-9 and CAS 51596-10-2), is described in DE 2329486 (1973). B-28, cyproflanilide (CAS 2375110-88-4), is described in WO 2020001067. B-29, thiocyclam (CAS 31895-21-3), is described in ZA 7007824. B-30, guadipyr (CAS 1227849-60-6), is described in WO 2010060231. B-31, 5-[[(1R,3R)-3-[3,5-bis(trifluoromethyl)phenyl]-2,2-dichloro-cyclopropanecarbonyl]amino]-2-chloro-N-[3-[(2,2-difluoro-acetyl)amino]-2,4-difluoro-phenyl]benzamide) (CAS 2220132-55-6) and B-32, 5-[[trans-3-[3,5-bis(trifluoromethyl)phenyl]-2,2-dichloro-cyclopropanecarbonyl]amino]-2-chloro-N-[3-[(2,2-difluoro-acetyl)amino]-2,4-difluoro-phenyl]benzamide are described in WO2018071327. B-33, afoxolaner (CAS 1093861-60-9), is described in WO 2009002809. B-34, fluralaner (CAS 864731-61-3), is described in WO 2005085216. B-35, sarolaner (CAS 1398609-39-6), is described in WO 2012120399. B-36, ivermectin (CAS 70288-86-7), is described in EP 146414 (1985). B-37, tigolaner (CAS 1621436-41-6), is described in WO 2014122083. B-38, afidopyropen (CAS 915972-17-7), is described in WO 2006129714. B-39, flonicamid (CAS 158062-67-0), is described in EP 580374 (1994). B-40, fluxametamide (CAS 928783-29-3), is described in WO 2007026965. B-41, spirotetramat (CAS 203313-25-1), is described in WO 9805638. B-42, triflumezopyrim (CAS 1263133-33-0), is described in WO 2011017351. B-43, broflanilide (CAS 1207727-04-5), is described in WO 2010018714. B-44, nicofluprole (CAS 1771741-86-6), is described in WO 2015067647. B-45, spinetoram (CAS 935545-74-7), is described in WO 2008057520. B-46, thiacloprid (CAS 111988-49-9), is described in EP 235725 (1987). B-47, flupyradifurone (CAS 951659-40-8), is described in DE 102006015467 (2007). B-48, imidacloprid (CAS 138261-41-3), is described in EP 192060 (1986). B-49, sulfoxaflor (CAS 946578-00-3), is described in WO 2007095229. B-50, acetamiprid (CAS 135410-20-7), is described in WO 9104965. B-51, nitenpyram (CAS 150824-47-8), is described in EP 392560 (1990). B-52, clothianidin (CAS 210880-92-5), is described in WO 9827074. B-53, thiamethoxam (CAS 153719-23-4), is described in EP 580553 (1994). B-54, tetraniliprole (CAS 1229654-66-3), is described in WO 2010069502. B-55, flubendiamide (CAS 272451-65-7), is described in EP 1006107 (2000). B-56, chlorantraniliprole (CAS 500008-45-7), is described in WO 2003015518. B-57, cyantraniliprole (CAS 736994-63-1), is described in WO 2004067528. B-58, oxazosulfyl (CAS 1403615-77-9), is described in WO 2014104407. B-59, indoxacarb (CAS 173584-44-6), is described in WO 9529171. B-60, cyfluthrin (CAS 68359-37-5), is described in DE 2709264 (1978). B-61, deltamethrin (CAS 52918-63-5), is described in DE 2439177 (1975). B-62, acrinathrin (CAS 101007-06-1), is described in U.S. Pat. No. 4,542,142 (1985). B-63, zeta-cypermethrin (CAS 1315501-18-8), is described in WO 2008005764. B-64, bifenthrin (CAS 82657-04-3), is described in GB 2085005 (1982). B-65, gamma-cyhalothrin (CAS 76703-62-3), is described in GB 2143823 (1985). B-66, alpha-cypermethrin (CAS 67375-30-8), is described in DE 2850502 (1979). B-67, permethrin (CAS 52645-53-1), is described in DE 2437882 (1975). B-68, fenpropathrin (CAS 39515-41-8), is described in DE 2231312 (1973). B-69, fenvalerate (CAS 51630-58-1), is described in DE 2335347 (1974). B-70, esfenvalerate (CAS 66230-04-4), is described in DE 2737297 (1978). B-71, lambda-cyhalothrin (CAS 91465-08-6), is described in EP 106469 (1984). B-112, pyrapropoyne (CAS 1803108-03-3), is described in WO 2015119246. B-136, cyflufenamid (CAS 180409-60-3), is described in WO 9619442. B-140, isocycloseram (CAS 2061933-85-3) is described in WO 2011067272. B-141, tefluthrin (CAS 79538-32-2) is described in JP 55111445 (1980). B-142, cyclobutrifluram (CAS 1644251-74-0) is described in WO 2015003951. B-143, dimpropyridaz (CAS 1403615-77-9) is described in WO 2012143317. B-144, fipronil (CAS 120068-37-3) is described in EP 295117 (1988). The remaining named, compounds are also known from the art.

Examples of the above mentioned pests are:
from the order Acarina, for example,
Acalitus spp, Aculus spp, Acaricalus spp, Aceria spp, Acarus siro, Amblyomma spp., Argas spp., Boophilus spp., Brevipalpus spp., Bryobia spp, Calipitrimerus spp., Chorioptes spp., Dermanyssus gallinae, Dermatophagoides spp, Eotetranychus spp, Eriophyes spp., Hemitarsonemus spp, Hyalomma spp., Ixodes spp., Olygonychus spp, Ornithodoros spp., Polyphagotarsone latus, Panonychus spp., Phyllocoptruta oleivora, Phytonemus spp, Polyphagotarsonemus spp, Psoroptes spp., Rhipicephalus spp., Rhizoglyphus spp., Sarcoptes spp., Steneotarsonemus spp, Tarsonemus spp. and Tetranychus spp.;
from the order Anoplura, for example,
Haematopinus spp., Linognathus spp., Pediculus spp., Pemphigus spp. and Phylloxera spp.;
from the order Coleoptera, for example,
Agriotes spp., Amphimallon majale, Anomala orientalis, Anthonomus spp., Aphodius spp, Astylus atromaculatus, Ataenius spp, Atomaria linearis, Chaetocnema tibialis, Cerotoma spp, Conoderus spp, Cosmopolites spp., Cotinis nitida, Curculio spp., Cyclocephala spp, Dermestes spp., Diabrotica spp., Diloboderus abderus, Epilachna spp., Eremnus spp., Heteronychus arator, Hypothenemus hampei, Lagria vilosa, Leptinotarsa decemlineata, Lissorhoptrus spp., Liogenys spp, Maecolaspis spp, Maladera castanea, Megascelis spp, Melighetes aeneus, Melolontha spp., Myochrous armatus, Orycaephilus spp., Otiorhynchus spp., Phyllophaga spp, Phlyctinus spp., Popillia spp., Psylliodes spp., Rhyssomatus aubtilis, Rhizopertha spp., Scarabeidae, Sitophilus spp., Sitotroga spp., Somaticus spp, Sphenophorus spp, Sternechus subsignatus, Tenebrio spp., Tribolium spp. and Trogoderma spp.;
from the order Diptera, for example,
Aedes spp., Anopheles spp, Antherigona soccata, Bactrocea oleae, Bibio hortulanus, Bradysia spp, Calliphora erythrocephala, Ceratitis spp., Chrysomyia spp., Culex spp., Cuterebra spp., Dacus spp., Delia spp, Drosophila melanogaster, Fannia spp., Gastrophilus spp., Geomyza tripunctata, Glossina spp., Hypoderma spp., Hyppobosca spp., Liriomyza spp., Lucilia spp., Melanagromyza spp., Musca spp., Oestrus spp., Orseolia spp., Oscinella frit, Pegomyia hyoscyami, Phorbia spp., Rhagoletis spp, Rivelia quadrifasciata, Scatella spp, Sciara spp., Stomoxys spp., Tabanus spp., Tannia spp. and Tipula spp.;
from the order Hemiptera, for example,
Acanthocoris scabrator, Acrosternum spp, Adelphocoris lineolatus, Aleurodes spp., Amblypelta nitida, Bathycoelia thalassina, Blissus spp, Cimex spp., Clavigralla tomentosicollis, Creontiades spp, Distantiella theobroma, Dichelops furcatus, Dysdercus spp., Edessa spp, Euchistus spp., Eurydema pulchrum, Eurygaster spp., Halyomorpha halys, Horcias nobilellus, Leptocorisa spp., Lygus spp, Margarodes spp, Murgantia histrionic, Neomegalotomus spp, Nesidiocoris tenuis, Nezara spp., Nysius simulans, Oebalus insularis, Piesma spp., Piezodorus spp, Rhodnius spp., Sahlbergella singularis, Scaptocoris castanea, Scotinophara spp., Thyanta spp, Triatoma spp., Vatiga illudens; Acyrthosium pisum, Adalges spp, Agalliana ensigera, Agonoscena targionii, Aleurodicus spp, Aleurocanthus spp, Aleurolobus barodensis, Aleurothrixus floccosus, Aleyrodes brassicae, Amarasca biguttula, Amritodus atkinsoni, Aonidiella spp., Aphididae, Aphis spp., Aspidiotus spp., Aulacorthum solani, Bactericera cockerelli, Bemisia spp, Brachycaudus spp, Brevicoryne brassicae, Cacopsylla spp, Cavariella aegopodii Scop., Ceroplaster spp., Chrysomphalus aonidium, Chrysomphalus dictyospermi, Cicadella spp, Cofana spectra, Cryptomyzus spp, Cicadulina spp, Coccus hesperidum, Dalbulus maidis, Dialeurodes spp, Diaphorina citri, Diuraphis noxia, Dysaphis spp, Empoasca spp., Eriosoma larigerum, Erythroneura spp., Gascardia spp., Glycaspis brimblecombei, Hyadaphis pseudobrassicae, Hyalopterus spp, Hyperomyzus pallidus, Idioscopus clypealis, Jacobiasca lybica, Laodelphax spp., Lecanium corni, Lepidosaphes spp., Lopaphis erysimi, Lyogenys maidis, Macrosiphum spp., Mahanarva spp, Metcalfa pruinosa, Metopolophium dirhodum, Myndus crudus, Myzus spp., Neotoxoptera sp, Nephotettix spp., Nilaparvata spp., Nippolachnus piri Mats, Odonaspis ruthae, Oregma lanigera Zehnter, Parabemisia myricae, Paratrioza cockerelli, Parlatoria spp., Pemphigus spp., Peregrinus maidis, Perkinsiella spp, Phorodon humuli, Phylloxera spp, Planococcus spp., Pseudaulacaspis spp., Pseudococcus spp., Pseudatomoscelis seriatus, Psylla spp., Pulvinaria aethiopica, Quadraspidiotus spp., Quesada gigas, Recilia dorsalis, Rhopalosiphum spp., Saissetia spp., Scaphoideus spp., Schizaphis spp., Sitobion spp., Sogatella furcifera, Spissistilus festinus, Tarophagus Proserpina, Toxoptera spp, Trialeurodes spp, Tridiscus sporoboli, Trionymus spp, Trioza erytreae, Unaspis citri, Zygina flammigera, Zyginidia scutellaris;
from the order Hymenoptera, for example,
Acromyrmex, Arge spp, Atta spp., Cephus spp., Diprion spp., Diprionidae, Gilpinia polytoma, Hoplo-campa spp., Lasius spp., Monomorium pharaonis, Neodiprion spp., Pogonomyrmex spp, Slenopsis invicta, Solenopsis spp. and Vespa spp.;
from the order Isoptera, for example,
Coptotermes spp, Corniternes cumulans, Incisitermes spp, Macrotermes spp, Mastotermes spp, Microtermes spp, Reticulitermes spp.; Solenopsis geminate;
from the order Lepidoptera, for example,
Acleris spp., Adoxophyes spp., Aegeria spp., Agrotis spp., Alabama argillaceae, Amylois spp., Anticarsia gemmatalis, Archips spp., Argyresthia spp, Argyrotaenia spp., Autographa spp., Bucculatrix thurberiella, Busseola fusca, Cadra cautella, Carposina nipponensis, Chilo spp., Choristoneura spp., Chrysoteuchia topiaria, Clysia ambiguella, Cnaphalocrocis spp., Cnephasia spp., Cochylis spp., Coleophora spp., Colias lesbia, Cosmophila flava, Crambus spp, Crocidolomia binotalis, Cryptophlebia leucotreta, Cydalima perspectalis, Cydia spp., Diaphania perspectalis, Diatraea spp., Diparopsis castanea, Earias spp., Elasmopalpus lignosellus, Eldana saccharina, Ephestia spp., Epinotia spp, Estigmene acrea, Etiella zinckinella, Eucosma spp., Eupoecilia ambiguella, Euproctis spp., Euxoa spp., Feltia jaculiferia, Grapholita spp., Hedya nubiferana, Heliothis spp., Hellula undalis, Herpetogramma spp, Hyphantria cunea, Keiferia lycopersicella, Lasmopalpus lignosellus, Leucoptera scitella, Lithocollethis spp., Lobesia botrana, Loxostege bifidalis, Lymantria spp., Lyonetia spp., Malacosoma spp., Mamestra brassicae, Manduca sexta, Mythimna spp, Noctua spp, Operophtera spp., Orniodes indica, Ostrinia nubilalis, Pammene spp., Pandemis spp., Panolis flammea, Papaipema nebris, Pectinophora gossypiela, Perileucoptera coffeella, Pseudaletia unipuncta, Phthorimaea operculella, Pieris rapae, Pieris spp., Plutella xylostella, Prays spp., Pseudoplusia spp, Rachiplusia nu, Richia albicosta, Scirpophaga spp., Sesamia spp., Sparganothis spp., Spodoptera spp., Sylepta derogate, Synanthedon spp., Thaumetopoea spp., Tortrix spp., Trichoplusia ni, Tuta absoluta, and Yponomeuta spp.;
from the order Mallophaga, for example,
Damalinea spp. and Trichodectes spp.;
from the order Orthoptera, for example,
Blatta spp., Blattella spp., Gryllotalpa spp., Leucophaea maderae, Locusta spp., Neocurtilla hexadactyla, Periplaneta spp., Scapteriscus spp, and Schistocerca spp.;
from the order Psocoptera, for example,
Liposcelis spp.;
from the order Siphonaptera, for example,
Ceratophyllus spp., Ctenocephalides spp. and Xenopsylla cheopis;
from the order Thysanoptera, for example,
Calliothrips phaseoli, Frankliniella spp., Heliothrips spp, Hercinothrips spp., Parthenothrips spp, Scirtothrips aurantii, Sericothrips variabilis, Taeniothrips spp., Thrips spp;
from the order Thysanura, for example, Lepisma saccharina.

In a further aspect, the invention may also relate to a method of controlling damage to plant and parts thereof by plant parasitic nematodes (Endoparasitic-, Semiendoparasitic- and Ectoparasitic nematodes), especially plant parasitic nematodes such as root knot nematodes, Meloidogyne hapla, Meloidogyne incognita, Meloidogyne javanica, Meloidogyne arenaria and other Meloidogyne species; cyst-forming nematodes, Globodera rostochiensis and other Globodera species; Heterodera avenae, Heterodera glycines, Heterodera schachtii, Heterodera trifolii, and other Heterodera species; Seed gall nematodes, Anguina species; Stem and foliar nematodes, Aphelenchoides species; Sting nematodes, Belonolaimus longicaudatus and other Belonolaimus species; Pine nematodes, Bursaphelenchus xylophilus and other Bursaphelenchus species; Ring nematodes, Criconema species, Criconemella species, Criconemoides species, Mesocriconema species; Stem and bulb nematodes, Ditylenchus destructor, Ditylenchus dipsaci and other Ditylenchus species; Awl nematodes, Dolichodorus species; Spiral nematodes, Heliocotylenchus multicinctus and other Helicotylenchus species; Sheath and sheathoid nematodes, Hemicycliophora species and Hemicriconemoides species; Hirshmanniella species; Lance nematodes, Hoploaimus species; false rootknot nematodes, Nacobbus species; Needle nematodes, Longidorus elongatus and other Longidorus species; Pin nematodes, Pratylenchus species; Lesion nematodes, Pratylenchus neglectus, Pratylenchus penetrans, Pratylenchus curvitatus, Pratylenchus goodeyi and other Pratylenchus species; Burrowing nematodes, Radopholus similis and other Radopholus species; Reniform nematodes, Rotylenchus robustus, Rotylenchus reniformis and other Rotylenchus species; Scutellonema species; Stubby root nematodes, Trichodorus primitivus and other Trichodorus species, Paratrichodorus species; Stunt nematodes, Tylenchorhynchus claytoni, Tylenchorhynchus dubius and other Tylenchorhynchus species; Citrus nematodes, Tylenchulus species; Dagger nematodes, Xiphinema species; and other plant parasitic nematode species, such as Subanguina spp., Hypsoperine spp., Macroposthonia spp., Melinius spp., Punctodera spp., and Quinisulcius spp.

The combinations and compositions of the invention may also have activity against the molluscs. Examples of which include, for example, Ampullariidae; Arion (A. ater, A. circumscriptus, A. hortensis, A. rufus); Bradybaenidae (Bradybaena fruticum); Cepaea (C. hortensis, C. Nemoralis); ochlodina; Deroceras (D. agrestis, D. empiricorum, D. laeve, D. reticulatum); Discus (D. rotundatus); Euomphalia; Galba (G. trunculata); Helicelia (H. itala, H. obvia); Helicidae Helicigona arbustorum); Helicodiscus; Helix (H. aperta); Limax (L. cinereoniger, L. flavus, L. marginatus, L. maximus, L. tenellus); Lymnaea; Milax (M. gagates, M. marginatus, M. sowerbyi); Opeas; Pomacea (P. canaticulata); Vallonia and Zanitoides.

The combinations and compositions according to the invention can be used for controlling, i. e. containing or destroying, pests of the abovementioned type which occur in particular on plants, especially on useful plants and ornamentals in agriculture, in horticulture and in forests, or on organs, such as fruits, flowers, foliage, stalks, tubers or roots, of such plants, and in some cases even plant organs which are formed at a later point in time remain protected against these pests.

Suitable target crops are, in particular, cereals, such as wheat, barley, rye, oats, rice, maize or sorghum; beet, such as sugar or fodder beet; fruit, for example pomaceous fruit, stone fruit or soft fruit, such as apples, pears, plums, peaches, almonds, cherries or berries, for example strawberries, raspberries or blackberries; leguminous crops, such as beans, lentils, peas or soya; oil crops, such as oilseed rape, mustard, poppies, olives, sunflowers, coconut, castor, cocoa or ground nuts; cucurbits, such as pumpkins, cucumbers or melons; fibre plants, such as cotton, flax, hemp or jute; citrus fruit, such as oranges, lemons, grapefruit or tangerines; vegetables, such as spinach, lettuce, asparagus, cabbages, carrots, onions, tomatoes, potatoes or bell peppers; Lauraceae, such as avocado, Cinnamonium or camphor; and also tobacco, nuts, coffee, eggplants, sugarcane, tea, pepper, grapevines, hops, the plantain family and latex plants.

The combinations and compositions of the present invention may be also used on any ornamental and/or vegetable crops, including flowers, shrubs, broad-leaved trees and evergreens.

For example the invention may be used on any of the following ornamental species: Ageratum spp., Alonsoa spp., Anemone spp., Anisodontea capsenisis, Anthemis spp., Antirrhinum spp., Aster spp., Begonia spp. (e.g. B. elatior, B. semperflorens, B. tubéreux), Bougainvillea spp., Brachycome spp., Brassica spp. (ornamental), Calceolaria spp., Capsicum annuum, Catharanthus roseus, Canna spp., Centaurea spp., Chrysanthemum spp., Cineraria spp. (C. maritime), Coreopsis spp., Crassula coccinea, Cuphea ignea, Dahlia spp., Delphinium spp., Dicentra spectabilis, Dorotheantus spp., Eustoma grandiflorum, Forsythia spp., Fuchsia spp., Geranium gnaphalium, Gerbera spp., Gomphrena globosa, Heliotropium spp., Helianthus spp., Hibiscus spp., Hortensia spp., Hydrangea spp., Hypoestes phyllostachya, Impatiens spp. (I. walleriana), Iresines spp., Kalanchoe spp., Lantana camara, Lavatera trimestris, Leonotis leonurus, Lilium spp., Mesembryanthemum spp., Mimulus spp., Monarda spp., Nemesia spp., Tagetes spp., Dianthus spp. (carnation), Canna spp., Oxalis spp., Bellis spp., Pelargonium spp. (P. peltatum, P. zonale), Viola spp. (pansy), Petunia spp., Phlox spp., Plecthranthus spp., Poinsettia spp., Parthenocissus spp. (P. quinquefolia, P. tricuspidata), Primula spp., Ranunculus spp., Rhododendron spp., Rosa spp. (rose), Rudbeckia spp., Saintpaulia spp., Salvia spp., Scaevola aemola, Schizanthus wisetonensis, Sedum spp., Solanum spp., Surfinia spp., Tagetes spp., Nicotinia spp., Verbena spp., Zinnia spp. and other bedding plants.

For example the invention may be used on any of the following vegetable species: Allium spp. (A. sativum, A. cepa, A. oschaninii, A. porrum, A. ascalonicum, A. fistulosum), Anthriscus cerefolium, Apium graveolus, Asparagus officinalis, Beta vulgarus, Brassica spp. (B. oleracea, B. pekinensis, B. rapa), Capsicum annuum, Cicer arietinum, Cichorium endivia, Cichorum spp. (C. intybus, C. endivia), Citrillus lanatus, Cucumis spp. (C. sativus, C. melo), Cucurbita spp. (C. pepo, C. maxima), Cyanara spp. (C. scolymus, C. cardunculus), Daucus carota, Foeniculum vulgare, Hypericum spp., Lactuca sativa, Lycopersicon spp. (L. esculentum, L. lycopersicum), Mentha spp., Ocimum basilicum, Petroselinum crispum, Phaseolus spp. (P. vulgaris, P. coccineus), Pisum sativum, Raphanus sativus, Rheum rhaponticum, Rosemarinus spp., Salvia spp., Scorzonera hispanica, Solanum melongena, Spinacea oleracea, Valerianella spp. (V. locusta, V. eriocarpa) and Vicia faba.

Preferred ornamental species include African violet, Begonia, Dahlia, Gerbera, Hydrangea, Verbena, Rosa, Kalanchoe, Poinsettia, Aster, Centaurea, Coreopsis, Delphinium, Monarda, Phlox, Rudbeckia, Sedum, Petunia, Viola, Impatiens, Geranium, Chrysanthemum, Ranunculus, Fuchsia, Salvia, Hortensia, rosemary, sage, St. Johnswort, mint, sweet pepper, tomato and cucumber.

The combinations and compositions of the invention are particularly suitable for control of
a pest of the order Hemiptera, for example, one or more of the species Bemisia tabaci, Aphis craccivora, Myzus persicae, Rhopalosiphum Padi, Nilaparvata lugens, and Euschistus heros (preferably in vegetables, soybeans, and sugarcane);
a pest of the order Lepidoptera, for example, one or more of the species Spodoptera littoralis, Spodoptera frugiperda, Plutella xylostella, Cnaphalocrocis medinalis, Cydia pomonella, Chrysodeixis includes, Chilo suppressalis, Elasmopalpus lignosellus, Pseudoplusia includens, and Tuta absoluta (preferably in rice, vegetables and corn);
a pest of the order Thysanoptera, such as the family Thripidae, for example, one or more of Thrips tabaci and Frankliniella occidentalis (preferably in vegetables); and
soil pests (such as of the order Coleoptera), for example, the species Diabrotica balteata, Agriotes spp. and Leptinotarsa decemlineata (preferably in vegetables and corn).

The term “crops” is to be understood as including also crop plants which have been so transformed by the use of recombinant DNA techniques that they are capable of synthesising one or more selectively acting toxins, such as are known, for example, from toxin-producing bacteria, especially those of the genus Bacillus.

Toxins that can be expressed by such transgenic plants include, for example, insecticidal proteins, for example insecticidal proteins from Bacillus cereus or Bacillus popilliae; or insecticidal proteins from Bacillus thuringiensis, such as δ-endotoxins, e.g. Cry1Ab, Cry1Ac, Cry1F, Cry1Fa2, Cry2Ab, Cry3A, Cry3Bb1 or Cry9C, or vegetative insecticidal proteins (Vip), e.g. Vip1, Vip2, Vip3 or Vip3A; or insecticidal proteins of bacteria colonising nematodes, for example Photorhabdus spp. or Xenorhabdus spp., such as Photorhabdus luminescens, Xenorhabdus nematophilus; toxins produced by animals, such as scorpion toxins, arachnid toxins, wasp toxins and other insect-specific neurotoxins; toxins produced by fungi, such as Streptomycetes toxins, plant lectins, such as pea lectins, barley lectins or snowdrop lectins; agglutinins; proteinase inhibitors, such as trypsin inhibitors, serine protease inhibitors, patatin, cystatin, papain inhibitors; ribosome-inactivating proteins (RIP), such as ricin, maize-RIP, abrin, luffin, saporin or bryodin; steroid metabolism enzymes, such as 3-hydroxysteroidoxidase, ecdysteroid-UDP-glycosyl-transferase, cholesterol oxidases, ecdysone inhibitors, HMG-COA-reductase, ion channel blockers, such as blockers of sodium or calcium channels, juvenile hormone esterase, diuretic hormone receptors, stilbene synthase, bibenzyl synthase, chitinases and glucanases.

In the context of the present invention there are to be understood by δ-endotoxins, for example Cry1Ab, Cry1Ac, Cry1F, Cry1Fa2, Cry2Ab, Cry3A, Cry3Bb1 or Cry9C, or vegetative insecticidal proteins (Vip), for example Vip1, Vip2, Vip3 or Vip3A, expressly also hybrid toxins, truncated toxins and modified toxins. Hybrid toxins are produced recombinantly by a new combination of different domains of those proteins (see, for example, WO 02/15701). Truncated toxins, for example a truncated Cry1Ab, are known. In the case of modified toxins, one or more amino acids of the naturally occurring toxin are replaced. In such amino acid replacements, preferably non-naturally present protease recognition sequences are inserted into the toxin, such as, for example, in the case of Cry3A055, a cathepsin-G-recognition sequence is inserted into a Cry3A toxin (see WO 03/018810).

Examples of such toxins or transgenic plants capable of synthesising such toxins are disclosed, for example, in EP-A-0 374 753, WO 93/07278, WO 95/34656, EP-A-0 427 529, EP-A-451 878 and WO 03/052073.

The processes for the preparation of such transgenic plants are generally known to the person skilled in the art and are described, for example, in the publications mentioned above. Cryl-type deoxyribonucleic acids and their preparation are known, for example, from WO 95/34656, EP-A-0 367 474, EP-A-0 401 979 and WO 90/13651.

The toxin contained in the transgenic plants imparts to the plants tolerance to harmful insects. Such insects can occur in any taxonomic group of insects, but are especially commonly found in the beetles (Coleoptera), two-winged insects (Diptera) and moths (Lepidoptera).

Transgenic plants containing one or more genes that code for an insecticidal resistance and express one or more toxins are known and some of them are commercially available. Examples of such plants are: YieldGard® (maize variety that expresses a Cry1Ab toxin); YieldGard Rootworm® (maize variety that expresses a Cry3Bb1 toxin); YieldGard Plus® (maize variety that expresses a Cry1Ab and a Cry3Bb1 toxin); Starlink® (maize variety that expresses a Cry9C toxin); Herculex I® (maize variety that expresses a Cry1Fa2 toxin and the enzyme phosphinothricine N-acetyltransferase (PAT) to achieve tolerance to the herbicide glufosinate ammonium); NuCOTN 33B® (cotton variety that expresses a Cry1Ac toxin); Bollgard I® (cotton variety that expresses a Cry1Ac toxin); Bollgard II® (cotton variety that expresses a Cry1Ac and a Cry2Ab toxin); VipCot® (cotton variety that expresses a Vip3A and a Cry1Ab toxin); NewLeaf® (potato variety that expresses a Cry3A toxin); NatureGard®, Agrisure® GT Advantage (GA21 glyphosate-tolerant trait), Agrisure® CB Advantage (Bt11 corn borer (CB) trait) and Protecta®.

Further examples of such transgenic crops are:

1. Bt11 Maize from Syngenta Seeds SAS, Chemin de l′Hobit 27, F-31 790 St. Sauveur, France, registration number C/FR/96/05/10. Genetically modified Zea mays which has been rendered resistant to attack by the European corn borer (Ostrinia nubilalis and Sesamia nonagrioides) by transgenic expression of a truncated Cry1Ab toxin. Bt11 maize also transgenically expresses the enzyme PAT to achieve tolerance to the herbicide glufosinate ammonium.

2. Bt176 Maize from Syngenta Seeds SAS, Chemin de l′Hobit 27, F-31 790 St. Sauveur, France, registration number C/FR/96/05/10. Genetically modified Zea mays which has been rendered resistant to attack by the European corn borer (Ostrinia nubilalis and Sesamia nonagrioides) by transgenic expression of a Cry1Ab toxin. Bt176 maize also transgenically expresses the enzyme PAT to achieve tolerance to the herbicide glufosinate ammonium.

3. MIR604 Maize from Syngenta Seeds SAS, Chemin de l′Hobit 27, F-31 790 St. Sauveur, France, registration number C/FR/96/05/10. Maize which has been rendered insect-resistant by transgenic expression of a modified Cry3A toxin. This toxin is Cry3A055 modified by insertion of a cathepsin-G-protease recognition sequence. The preparation of such transgenic maize plants is described in WO 03/018810.

4. MON 863 Maize from Monsanto Europe S. A. 270-272 Avenue de Tervuren, B-1150 Brussels, Belgium, registration number C/DE/02/9. MON 863 expresses a Cry3Bb1 toxin and has resistance to certain Coleoptera insects.

5. IPC 531 Cotton from Monsanto Europe S. A. 270-272 Avenue de Tervuren, B-1150 Brussels, Belgium, registration number C/ES/96/02.

6. 1507 Maize from Pioneer Overseas Corporation, Avenue Tedesco, 7 B-1160 Brussels, Belgium, registration number C/NL/00/10. Genetically modified maize for the expression of the protein Cry1F for achieving resistance to certain Lepidoptera insects and of the PAT protein for achieving tolerance to the herbicide glufosinate ammonium.

7. NK603×MON 810 Maize from Monsanto Europe S. A. 270-272 Avenue de Tervuren, B-1150 Brussels, Belgium, registration number C/GB/02/M3/03. Consists of conventionally bred hybrid maize varieties by crossing the genetically modified varieties NK603 and MON 810. NK603×MON 810 Maize transgenically expresses the protein CP4 EPSPS, obtained from Agrobacterium sp. strain CP4, which imparts tolerance to the herbicide Roundup® (contains glyphosate), and also a Cry1Ab toxin obtained from Bacillus thuringiensis subsp. kurstaki which brings about tolerance to certain Lepidoptera, include the European corn borer.

Transgenic crops of insect-resistant plants are also described in BATS (Zentrum für Biosicherheit und Nachhaltigkeit, Zentrum BATS, Clarastrasse 13, 4058 Basel, Switzerland) Report 2003, (http://bats.ch).

The term “crops” is to be understood as including also crop plants which have been so transformed by the use of recombinant DNA techniques that they are capable of synthesising antipathogenic substances having a selective action, such as, for example, the so-called “pathogenesis-related proteins” (PRPs, see e.g. EP-A-0 392 225). Examples of such antipathogenic substances and transgenic plants capable of synthesising such antipathogenic substances are known, for example, from EP-A-0 392 225, WO 95/33818 and EP-A-0 353 191. The methods of producing such transgenic plants are generally known to the person skilled in the art and are described, for example, in the publications mentioned above.

Crops may also be modified for enhanced resistance to fungal (for example Fusarium, Anthracnose, or Phytophthora), bacterial (for example Pseudomonas) or viral (for example potato leafroll virus, tomato spotted wilt virus, cucumber mosaic virus) pathogens.

Crops also include those that have enhanced resistance to nematodes, such as the soybean cyst nematode.

Crops that are tolerance to abiotic stress include those that have enhanced tolerance to drought, high salt, high temperature, chill, frost, or light radiation, for example through expression of NF-YB or other proteins known in the art.

Antipathogenic substances which can be expressed by such transgenic plants include, for example, ion channel blockers, such as blockers for sodium and calcium channels, for example the viral KP1, KP4 or KP6 toxins; stilbene synthases; bibenzyl synthases; chitinases; glucanases; the so-called “pathogenesis-related proteins” (PRPs; see e.g. EP-A-0 392 225); antipathogenic substances produced by microorganisms, for example peptide antibiotics or heterocyclic antibiotics (see e.g. WO 95/33818) or protein or polypeptide factors involved in plant pathogen defence (so-called “plant disease resistance genes”, as described in WO 03/000906).

Further areas of use of the compositions according to the invention are the protection of stored goods and store rooms and the protection of raw materials, such as wood, textiles, floor coverings or buildings, and also in the hygiene sector, especially the protection of humans, domestic animals and productive livestock against pests of the mentioned type.

The present invention provides a method of improving the tolerance of a plant to abiotic stress, wherein the method comprises applying to the plant, plant part, plant propagation material, or plant growing locus a composition as described herein.

The present invention provides a method for regulating or improving the growth of a plant, wherein the method comprises applying to the plant, plant part, plant propagation material, or plant growing locus a composition as described herein. In one embodiment, plant growth is regulated or improved when the plant is subject to abiotic stress conditions.

The term “regulating or improving the growth of a crop” means an improvement in plant vigour, an improvement in plant quality, improved tolerance to stress factors, and/or improved input use efficiency.

The term “plants” refers to all physical parts of a plant, including seeds, seedlings, saplings, roots, tubers, stems, stalks, foliage, and fruits.

The term “locus” as used herein means fields in or on which plants are growing, or where seeds of cultivated plants are sown, or where seed will be placed into the soil. It includes soil, seeds, and seedlings, as well as established vegetation.

The term “plant propagation material” denotes all generative parts of a plant, for example seeds or vegetative parts of plants such as cuttings and tubers. It includes seeds in the strict sense, as well as roots, fruits, tubers, bulbs, rhizomes, and parts of plants.

Where a range of numbers is disclosed herein (for example, 1 to 10), this is intended to include all numbers and intervening values within that range (for example, 1, 1.1, 2, 3, 3.9, 4, 5, 6, 6.5, 7, 8, 9 and 10) and also any sub-range of numbers and intervening values within that range (for example, 2 to 8, 1.5 to 5.5 and 3.1 to 4.7). Additionally, it is intended that the both the upper and lower limits specified are included within the range.

Where ranges or values used herein are preceded by the term “about”, this term is intended to provide support for both the exact number that it precedes, and also a number that is near to or approximately the number that it precedes. In determining whether a number is near to or approximately a specifically recited number, the near or approximating number may be a number, which would be rounded to or be substantially equivalent to the specifically recited number. For example, the term “about 5” includes 5.0, 4.5, 5.4, 4.92, 5.01, and so on.

The composition can be in the form of concentrates which are diluted prior to use, although ready-to-use compositions can also be made. The final dilution is usually made with water, but can be made instead of, or in addition to, water, with, for example, liquid fertilisers, micronutrients, biological organisms, oil or solvents.

The compositions according to the invention are generally formulated in various ways using formulation adjuvants, such as carriers, solvents and surface-active substances. The formulations can be in various physical forms, e.g. in the form of dusting powders, gels, wettable powders, water-dispersible granules, water-dispersible tablets, effervescent pellets, emulsifiable concentrates, micro-emulsifiable concentrates, oil-in-water emulsions, oil-flowables, aqueous dispersions, oily dispersions, suspo-emulsions, capsule suspensions, emulsifiable granules, soluble liquids, water-soluble concentrates (with water or a water-miscible organic solvent as carrier), impregnated polymer films or in other forms known e.g. from the Manual on Development and Use of FAO and WHO Specifications for Pesticides, United Nations, First Edition, Second Revision (2010). Such formulations can either be used directly or diluted prior to use. The dilutions can be made, for example, with water, liquid fertilisers, micronutrients, biological organisms, oil or solvents.

The formulations can be prepared e.g. by mixing the active ingredient with the formulation adjuvants in order to obtain compositions in the form of finely divided solids, granules, solutions, dispersions or emulsions. The active ingredients can also be formulated with other adjuvants, such as finely divided solids, mineral oils, oils of vegetable or animal origin, modified oils of vegetable or animal origin, organic solvents, water, surface-active substances or combinations thereof.

The active ingredients can also be contained in very fine microcapsules. Microcapsules contain the active ingredients in a porous carrier. This enables the active ingredients to be released into the environment in controlled amounts (e.g. slow-release). Microcapsules usually have a diameter of from 0.1 to 500 microns. They contain active ingredients in an amount of about from 25 to 95% by weight of the capsule weight. The active ingredients can be in the form of a monolithic solid, in the form of fine particles in solid or liquid dispersion or in the form of a suitable solution. The encapsulating membranes can comprise, for example, natural or synthetic rubbers, cellulose, styrene/butadiene copolymers, polyacrylonitrile, polyacrylate, polyesters, polyamides, polyureas, polyurethane or chemically modified polymers and starch xanthates or other polymers that are known to the person skilled in the art. Alternatively, very fine microcapsules can be formed in which the active ingredient is contained in the form of finely divided particles in a solid matrix of base substance, but the microcapsules are not themselves encapsulated.

The formulation adjuvants that are suitable for the preparation of the compositions according to the invention are known per se. As liquid carriers there may be used: water, toluene, xylene, petroleum ether, vegetable oils, acetone, methyl ethyl ketone, cyclohexanone, acid anhydrides, acetonitrile, acetophenone, amyl acetate, 2-butanone, butylene carbonate, chlorobenzene, cyclohexane, cyclohexanol, alkyl esters of acetic acid, diacetone alcohol, 1,2-dichloropropane, diethanolamine, p-diethylbenzene, diethylene glycol, diethylene glycol abietate, diethylene glycol butyl ether, diethylene glycol ethyl ether, diethylene glycol methyl ether, N,N-dimethylformamide, dimethyl sulfoxide, 1,4-dioxane, dipropylene glycol, dipropylene glycol methyl ether, dipropylene glycol dibenzoate, diproxitol, alkylpyrrolidone, ethyl acetate, 2-ethylhexanol, ethylene carbonate, 1,1,1-trichloroethane, 2-heptanone, alpha-pinene, d-limonene, ethyl lactate, ethylene glycol, ethylene glycol butyl ether, ethylene glycol methyl ether, gamma-butyrolactone, glycerol, glycerol acetate, glycerol diacetate, glycerol triacetate, hexadecane, hexylene glycol, isoamyl acetate, isobornyl acetate, isooctane, isophorone, isopropylbenzene, isopropyl myristate, lactic acid, laurylamine, mesityl oxide, methoxypropanol, methyl isoamyl ketone, methyl isobutyl ketone, methyl laurate, methyl octanoate, methyl oleate, methylene chloride, m-xylene, n-hexane, n-octylamine, octadecanoic acid, octylamine acetate, oleic acid, oleylamine, o-xylene, phenol, polyethylene glycol, propionic acid, propyl lactate, propylene carbonate, propylene glycol, propylene glycol methyl ether, p-xylene, toluene, triethyl phosphate, triethylene glycol, xylenesulfonic acid, paraffin, mineral oil, trichloroethylene, perchloroethylene, ethyl acetate, amyl acetate, butyl acetate, propylene glycol methyl ether, diethylene glycol methyl ether, methanol, ethanol, isopropanol, and alcohols of higher molecular weight, such as amyl alcohol, tetrahydro-furfuryl alcohol, hexanol, octanol, ethylene glycol, propylene glycol, glycerol, N-methyl-2-pyrrolidone and the like.

Suitable solid carriers are, for example, talc, titanium dioxide, pyrophyllite clay, silica, attapulgite clay, kieselguhr, limestone, calcium carbonate, bentonite, calcium montmorillonite, cottonseed husks, wheat flour, soybean flour, pumice, wood flour, ground walnut shells, lignin and similar substances.

A large number of surface-active substances can advantageously be used in both solid and liquid formulations, especially in those formulations which can be diluted with a carrier prior to use. Surface-active substances may be anionic, cationic, non-ionic or polymeric and they can be used as emulsifiers, wetting agents or suspending agents or for other purposes. Typical surface-active substances include, for example, salts of alkyl sulfates, such as diethanolammonium lauryl sulfate
salts of alkylarylsulfonates, such as calcium dodecyl-benzenesulfonate
alkylphenol/alkylene oxide addition products, such as nonylphenol ethoxylate
alcohol/alkylene oxide addition products, such as tridecylalcohol ethoxylate
soaps, such as sodium stearate
salts of alkylnaphthalenesulfonates, such as sodium dibutylnaphthalenesulfonate
dialkyl esters of sulfosuccinate salts, such as sodium di(2-ethylhexyl)sulfosuccinate
sorbitol esters, such as sorbitol oleate
quaternary amines, such as lauryltrimethylammonium chloride, polyethylene glycol esters of fatty acids, such as polyethylene glycol stearate
block copolymers of ethylene oxide and propylene oxide
and salts of mono and di-alkylphosphate esters
and also further substances described e.g. in Mccutcheon's Detergents and Emulsifiers Annual, MC Publishing Corp., Ridgewood New Jersey (1981).

Further adjuvants that can be used in pesticidal formulations include crystallisation inhibitors, viscosity modifiers, suspending agents, dyes, anti-oxidants, foaming agents, light absorbers, mixing auxiliaries, antifoams, complexing agents, neutralising or pH-modifying substances and buffers, corrosion inhibitors, fragrances, wetting agents, take-up enhancers, micro-nutrients, plasticisers, glidants, lubricants, dispersants, thickeners, antifreezes, microbicides, and liquid and solid fertilisers.

The compositions according to the invention can include an additive comprising an oil of vegetable or animal origin, a mineral oil, alkyl esters of such oils or mixtures of such oils and oil derivatives. The amount of oil additive in the composition according to the invention is generally from 0.01 to 10%, based on the mixture to be applied. For example, the oil additive can be added to a spray tank in the desired concentration after a spray mixture has been prepared. Preferred oil additives comprise mineral oils or an oil of vegetable origin, for example rapeseed oil, olive oil or sunflower oil, emulsified vegetable oil, alkyl esters of oils of vegetable origin, for example the methyl derivatives, or an oil of animal origin, such as fish oil or beef tallow. Preferred oil additives comprise alkyl esters of C8 C22 fatty acids, especially the methyl derivatives of C12-C18 fatty acids, for example the methyl esters of lauric acid, palmitic acid and oleic acid (methyl laurate, methyl palmitate and methyl oleate, respectively). Many oil derivatives are known from the Compendium of Herbicide Adjuvants, 10th Edition, Southern Illinois University, 2010.

The inventive compositions generally comprise from 0.1 to 99% by weight, especially from 0.1 to 95% by weight, of active ingredients and from 1 to 99.9% by weight of a formulation adjuvant which preferably includes from 0 to 25% by weight of a surface-active substance. Whereas commercial products may preferably be formulated as concentrates, the end user will normally employ dilute formulations.

The rates of application vary within wide limits and depend on the nature of the soil, the method of application, the crop plant, the pest to be controlled, the prevailing climatic conditions, and other factors governed by the method of application, the time of application and the target crop. As a general guideline active ingredients may be applied at a rate of from 1 to 2000 g/ha, especially from 10 to 1000 g/ha.

In certain examples, the rate of application of component (A) is from 20 to 200 g/ha. In some examples, the rate of application of component (A) is from 50 to 150 g/ha, optionally from 50 to 70 g/ha or about 100 g/ha.

In certain examples, the rate of application of component (B) is from 20 to 200 g/ha.

Preferred formulations can have the following compositions (weight %):

### Emulsifiable Concentrates:

active ingredient: 1 to 95%, preferably 60 to 90%
surface-active agent: 1 to 30%, preferably 5 to 20%
liquid carrier: 1 to 80%, preferably 1 to 35%

### Dusts:

active ingredient: 0.1 to 10%, preferably 0.1 to 5%
solid carrier: 99.9 to 90%, preferably 99.9 to 99%

### Suspension Concentrates:

active ingredient: 5 to 75%, preferably 10 to 50%
water: 94 to 24%, preferably 88 to 30%
surface-active agent: 1 to 40%, preferably 2 to 30%

### Wettable Powders:

active ingredient: 0.5 to 90%, preferably 1 to 80%
surface-active agent: 0.5 to 20%, preferably 1 to 15%
solid carrier: 5 to 95%, preferably 15 to 90%

### Granules:

active ingredient: 0.1 to 30%, preferably 0.1 to 15%
solid carrier: 99.5 to 70%, preferably 97 to 85%

The following Examples further illustrate, but do not limit, the invention.

| Wettable powders | a) | b) | c) |
|---|---|---|---|
| active ingredients | 25% | 50% | 75% |
| sodium lignosulfonate | 5% | 5% | — |
| sodium lauryl sulfate | 3% | — | 5% |
| sodium diisobutylnaphthalenesulfonate | — | 6% | 10% |
| phenol polyethylene glycol ether | — | 2% | — |
| (7-8 mol of ethylene oxide) | | | |
| highly dispersed silicic acid | 5% | 10% | 10% |
| Kaolin | 62% | 27% | — |

The combination is thoroughly mixed with the adjuvants and the mixture is thoroughly ground in a suitable mill, affording wettable powders that can be diluted with water to give suspensions of the desired concentration.

| Powders for dry seed treatment | a) | b) | c) |
|---|---|---|---|
| active ingredients | 25% | 50% | 75% |
| light mineral oil | 5% | 5% | 5% |
| highly dispersed silicic acid | 5% | 5% | — |
| Kaolin | 65% | 40% | — |
| Talcum | — | | 20 |

The combination is thoroughly mixed with the adjuvants and the mixture is thoroughly ground in a suitable mill, affording powders that can be used directly for seed treatment.

| Emulsifiable concentrate | |
|---|---|
| active ingredients | 10% |
| octylphenol polyethylene glycol ether | 3% |
| (4-5 mol of ethylene oxide) | |
| calcium dodecylbenzenesulfonate | 3% |
| castor oil polyglycol ether (35 mol of ethylene oxide) | 4% |
| Cyclohexanone | 30% |
| xylene mixture | 50% |

Emulsions of any required dilution, which can be used in plant protection, can be obtained from this concentrate by dilution with water.

| Dusts | a) | b) | c) |
|---|---|---|---|
| Active ingredients | 5% | 6% | 4% |
| Talcum | 95% | — | — |
| Kaolin | — | 94% | — |
| mineral filler | — | | 96% |

Ready-for-use dusts are obtained by mixing the combination with the carrier and grinding the mixture in a suitable mill. Such powders can also be used for dry dressings for seed.

| Extruder granules | |
|---|---|
| Active ingredients | 15% |
| sodium lignosulfonate | 2% |
| carboxymethylcellulose | 1% |
| Kaolin | 82% |

The combination is mixed and ground with the adjuvants, and the mixture is moistened with water. The mixture is extruded and then dried in a stream of air.

| Coated granules | |
|---|---|
| Active ingredients | 8% |
| polyethylene glycol (mol. wt. 200) | 3% |
| Kaolin | 89% |

The finely ground combination is uniformly applied, in a mixer, to the kaolin moistened with polyethylene glycol. Non-dusty coated granules are obtained in this manner.

### Suspension Concentrate

| | |
|---|---|
| active ingredients | 40% |
| propylene glycol | 10% |
| nonylphenol polyethylene glycol ether | 6% |
| (15 mol of ethylene oxide) | |
| Sodium lignosulfonate | 10% |
| carboxymethylcellulose | 1% |
| silicone oil (in the form of a 75% | 1% |
| emulsion in water) | |
| Water | 32% |

The finely ground combination is intimately mixed with the adjuvants, giving a suspension concentrate from which suspensions of any desired dilution can be obtained by dilution with water. Using such dilutions, living plants as well as plant propagation material can be treated and protected against infestation by microorganisms, by spraying, pouring or immersion.

### Flowable Concentrate for Seed Treatment

| | |
|---|---|
| active ingredients | 40% |
| propylene glycol | 5% |
| copolymer butanol PO/EO | 2% |
| Tristyrenephenole with 10-20 moles EO | 2% |
| 1,2-benzisothiazolin-3-one (in the form of | 0.5% |
| a 20% solution in water) | |
| monoazo-pigment calcium salt | 5% |
| Silicone oil (in the form of a 75% | 0.2% |
| emulsion in water) | |
| Water | 45.3% |

The finely ground combination is intimately mixed with the adjuvants, giving a suspension concentrate from which suspensions of any desired dilution can be obtained by dilution with water. Using such dilutions, living plants as well as plant propagation material can be treated and protected against infestation by microorganisms, by spraying, pouring or immersion.

### Slow Release Capsule Suspension

28 parts of the combination are mixed with 2 parts of an aromatic solvent and 7 parts of toluene diisocyanate/polymethylene-polyphenylisocyanate-mixture (8:1). This mixture is emulsified in a mixture of 1.2 parts of polyvinylalcohol, 0.05 parts of a defoamer and 51.6 parts of water until the desired particle size is achieved. To this emulsion a mixture of 2.8 parts 1,6-diaminohexane in 5.3 parts of water is added. The mixture is agitated until the polymerization reaction is completed. The obtained capsule suspension is stabilized by adding 0.25 parts of a thickener and 3 parts of a dispersing agent. The capsule suspension formulation contains 28% of the active ingredients. The medium capsule diameter is 8-15 microns. The resulting formulation is applied to seeds as an aqueous suspension in an apparatus suitable for that purpose.

The combination or composition of the present invention may be applied to a plant, part of the plant, plant organ, plant propagation material or a plant growing locus.

The application is generally made by spraying (A) and (B) separately (i.e the combination) or (A) and (B) together (i.e the composition), typically by tractor mounted sprayer for large areas, but other methods such as dusting (for powders), drip or drench can also be used. Alternatively the combination or composition may be applied in furrow or directly to a seed before or at the time of planting.

The combination or composition of the present invention may be applied pre-emergence or post-emergence. Where the combination or composition is used to regulate the growth of crop plants or enhance the tolerance to abiotic stress, it may be applied post-emergence of the crop. Where the combination or composition is used to inhibit or delay the germination of seeds, it may be applied pre-emergence. Where the combination or composition is used to control pests, it may be applied as a preventative (before pest establishment) or curative (after pest establishment) treatment.

The present invention envisages application of the combinations and compositions of the invention to plant propagation material prior to, during, or after planting, or any combination of these.

Although active ingredients can be applied to plant propagation material in any physiological state, a common approach is to use seeds in a sufficiently durable state to incur no damage during the treatment process. Typically, seed would have been harvested from the field removed from the plant and separated from any cob, stalk, outer husk, and surrounding pulp or other non-seed plant material. Seed would preferably also be biologically stable to the extent that treatment would not cause biological damage to the seed. It is believed that treatment can be applied to seed at any time between seed harvest and sowing of seed including during the sowing process.

Methods for applying or treating active ingredients on to plant propagation material or to the locus of planting are known in the art and include dressing, coating, pelleting and soaking as well as nursery tray application, in furrow application, soil drenching, soil injection, drip irrigation, application through sprinklers or central pivot, or incorporation into soil (broad cast or in band). Alternatively or in addition active ingredients may be applied on a suitable substrate sown together with the plant propagation material.

The rates of application of combinations and compositions of the present invention may vary within wide limits and depend on the nature of the soil, the method of application (pre- or post-emergence, seed dressing, application to the seed furrow, no tillage application etc.), the crop plant, the prevailing climatic conditions, and other factors governed by the method of application, the time of application and the target crop. For foliar or drench application, the combinations and compositions of the present invention are generally applied at a rate of from 1 to 2000 g/ha, especially from 5 to 1000 g/ha. For seed treatment the rate of application is generally between 0.0005 and 150 g per 100 kg of seed.

The combinations and compositions according to the invention can be used in combination with other pesticides, including other pesticides such as insecticides, acaricides, nematicides, fungicides, or agents that enhance the activity of the composition according to the invention, in for example chemical treatment or pest control programs. The combination may have further surprising advantages, which could be described as synergistic effects.

Suitable other pesticides are, for example, pesticides of the following classes of active ingredients: organophosphates, nitrophenol derivatives, thioureas, juvenile hormones, formamidines, benzophenone derivatives, ureas, pyrrole derivatives, carbamates, pyrethroids, chlorinated hydrocarbons, acylureas, pyridylmethyleneamino derivatives, macrolides, benzoylureas, neonicotinoids and biological agents such as Bacillus thurigiensis strains or bacterially-derived pesticides such as spinosads, avermectins and Cry proteins.

The compositions of the present invention may be applied to dicotyledonous or monocotyledonous crops. Crops of useful plants in which the composition according to the invention can be used include perennial and annual crops, such as berry plants for example blackberries, blueberries, cranberries, raspberries and strawberries, cereals for example barley, maize (corn), millet, oats, rice, rye, sorghum triticale and wheat, fibre plants for example cotton, flax, hemp, jute and sisal, field crops for example sugar and fodder beet, coffee, hops, mustard, oilseed rape (canola), poppy, sugar cane, sunflower, tea and tobacco, fruit trees for example apple, apricot, avocado, banana, cherry, citrus, nectarine, peach, pear and plum, grasses for example Bermuda grass, bluegrass, bentgrass, centipede grass, fescue, ryegrass, St. Augustine grass and Zoysia grass, herbs such as basil, borage, chives, coriander, lavender, lovage, mint, oregano, parsley, rosemary, sage and thyme, legumes for example beans, lentils, peas and soya beans, nuts for example almond, cashew, ground nut, hazelnut, peanut, pecan, pistachio and walnut, palms for example oil palm, ornamentals for example flowers, shrubs and trees, other trees, for example cacao, coconut, olive and rubber, vegetables for example asparagus, aubergine, broccoli, cabbage, carrot, cucumber, garlic, lettuce, marrow, melon, okra, onion, pepper, potato, pumpkin, rhubarb, spinach and tomato, and vines for example grapes.

Crops are to be understood as being those which are naturally occurring, obtained by conventional methods of breeding, or obtained by genetic engineering. They include crops which contain so-called output traits (e.g. improved storage stability, higher nutritional value and improved flavour).

Crops are to be understood as also including those crops which have been rendered tolerant to herbicides like bromoxynil or classes of herbicides such as ALS-, EPSPS-, GS-, HPPD- and PPO-inhibitors. An example of a crop that has been rendered tolerant to imidazolinones, e.g. imazamox, by conventional methods of breeding is Clearfield® summer canola. Examples of crops that have been rendered tolerant to herbicides by genetic engineering methods include e.g. glyphosate- and glufosinate-resistant maize varieties commercially available under the trade names RoundupReady®, Herculex I® and LibertyLink®.

Crops are also to be understood as being those which naturally are or have been rendered resistant to harmful insects. This includes plants transformed by the use of recombinant DNA techniques, for example, to be capable of synthesising one or more selectively acting toxins, such as are known, for example, from toxin-producing bacteria. Examples of toxins which can be expressed include d-endotoxins, vegetative insecticidal proteins (Vip), insecticidal proteins of bacteria colonising nematodes, and toxins produced by scorpions, arachnids, wasps and fungi.

An example of a crop that has been modified to express the Bacillus thuringiensis toxin is the Bt maize KnockOut (Syngenta Seeds). An example of a crop comprising more than one gene that codes for insecticidal resistance and thus expresses more than one toxin is VipCot® (Syngenta Seeds). Crops or seed material thereof can also be resistant to multiple types of pests (so-called stacked transgenic events when created by genetic modification). For example, a plant can have the ability to express an insecticidal protein while at the same time being herbicide tolerant, for example Herculex I® (Dow AgroSciences, Pioneer Hi-Bred International).

Normally, in the management of a crop a grower would use one or more other agronomic chemicals or biologicals in addition to the composition of the present invention.

The present invention also provides the use of the combination or composition as defined above for controlling insect and/or acarina and/or nematode pests.

In accordance with certain aspects of the present invention described above, the combination of component (A) selected from A-1, A-2, A-3, A-4 and A-5 and a component (B) selected from compounds B-33 to B-37 is useful in controlling parasites in or on an animal. The present invention further provides the use of such combinations in controlling endoparasites in and ectoparasites on an animal.

The term “controlling” when used in context of parasites in or on an animal refers to reducing the number of pests or parasites, eliminating pests or parasites and/or preventing further pest or parasite infestation.

The term “treating” when used in context of parasites in or on an animal refers to restraining, slowing, stopping or reversing the progression or severity of an existing symptom or disease.

The term “preventing” when used in context of parasites in or on an animal refers to the avoidance of a symptom or disease developing in the animal.

The term “animal” when used in context of parasites in or on an animal may refer to a mammal and a non-mammal, such as a bird or fish. In the case of a mammal, it may be a human or non-human mammal. Non-human mammals include, but are not limited to, livestock animals and companion animals. Livestock animals include, but are not limited to, cattle, camellids, pigs, sheep, goats and horses. Companion animals include, but are not limited to, dogs, cats and rabbits.

A “parasite” is a pest which lives in or on the host animal and benefits by deriving nutrients at the host animal's expense. An “endoparasite” is a parasite which lives in the host animal. An “ectoparasite” is a parasite which lives on the host animal. Ectoparasites include, but are not limited to, acari, insects, nematodes and crustaceans (e.g. sea lice). The Acari (or Acarina) sub-class comprises ticks and mites. Ticks include, but are not limited to, members of the following genera: Rhipicaphalus, for example, Rhipicaphalus (Boophilus) microplus and Rhipicephalus sanguineus; Amblyomrna; Dermacentor; Haemaphysalis; Hyalomma; Ixodes; Rhipicentor; Margaropus; Argas; Otobius; and Ornithodoros. Mites include, but are not limited to, members of the following genera: Chorioptes, for example Chorioptes bovis; Psoroptes, for example Psoroptes ovis; Cheyletiella; Dermanyssus; for example Dermanyssus gallinae; Ortnithonyssus; Demodex, for example Demodex canis; Sarcoptes, for example Sarcoptes scabiei; and Psorergates. Insects include, but are not limited to, members of the orders: Siphonaptera, Diptera, Phthiraptera, Lepidoptera, Coleoptera and Homoptera. Members of the Siphonaptera order include, but are not limited to, Ctenocephalides felis and Ctenocephatides canis. Members of the Diptera order include, but are not limited to, Musca spp.; bot fly, for example Gasterophilus intestinalis and Oestrus ovis; biting flies; horse flies, for example Haematopota spp. and Tabunus spp.; haematobia, for example haematobia irritans; Stomoxys; Lucilia; midges; and mosquitoes. Members of the Phthiraptera class include, but are not limited to, blood sucking lice and chewing lice, for example Bovicola ovis and Bovicola bovis.

The term “effective amount” when used in context of parasites in or on an animal refers to the amount or dose of the compound of the invention, or a salt thereof, which, upon single or multiple dose administration to the animal, provides the desired effect in or on the animal. The effective amount can be readily determined by the attending diagnostician, as one skilled in the art, by the use of known techniques and by observing results obtained under analogous circumstances. In determining the effective amount a number of factors are considered by the attending diagnostician, including, but not limited to: the species of mammal; its size, age, and general health; the parasite to be controlled and the degree of infestation; the specific disease or disorder involved; the degree of or involvement or the severity of the disease or disorder; the response of the individual; the particular compound administered; the mode of administration; the bioavailability characteristics of the preparation administered; the dose regimen selected; the use of concomitant medication; and other relevant circumstances.

The combinations of the invention may be administered to the animal by any route which has the desired effect including, but not limited to topically, orally, parenterally′ and subcutaneously. Topical administration is preferred. Formulations suitable for topical administration include, for example, solutions, emulsions and suspensions and may take the form of a pour-on, spot-on, spray-on, spray race or dip. In the alternative, the compounds of the invention may be administered by means of an ear tag or collar.

Salt forms of the compounds of the invention include both pharmaceutically acceptable salts and veterinary acceptable salts, which can be different to agrochemically acceptable salts.

Pharmaceutically and veterinary acceptable salts and common methodology for preparing them are well known in the art. See, for example, Gould, P. L., “Salt selection for basic drugs”, International Journal of Pharmaceutics, 33: 201-217 (1986); Bastin, R. J., et al. “Salt Selection and Optimization Procedures for Pharmaceutical New Chemical Entities”, Organic Process Research and Development, 4: 427-435 (2000); and Berge, S. M., et al., “Pharmaceutical Salts”, Journal of Pharmaceutical Sciences, 66: 1-19, (1977). One skilled in the art of synthesis will appreciate that the compounds of the invention are readily converted to and may be isolated as a salt, such as a hydrochloride salt, using techniques and conditions well known to one of ordinary skill in the art. In addition, one skilled in the art of synthesis will appreciate that the compounds of the invention are readily converted to and may be isolated as the corresponding free base from the corresponding salt.

The term “seed treatment” generally refers to application of a material to a seed prior to or during the time it is planted in soil to improve the handling characteristics of the seed, protect the seed prior to germination, support the germination and/or support the growth of the resulting plant. Some seed treatments are employed solely for the purpose of improving the handling characteristics or other physical characteristics of seeds, and include no agricultural active ingredients. Other seed treatments bind one or more active ingredients to seeds for various beneficial purposes. For example, seed treatments that include one or more active ingredients are commonly used to ensure uniform stand establishment by protecting against soilborne diseases and insects. Typical examples include the application of pesticides such as fungicides, insecticides and plant growth regulators. Systemic seed treatments may eliminate, or at least reduce the need for, traditional broadcast sprays of foliar fungicides or insecticides for certain early season airborne diseases and insects.

The seed treatment mixture can also comprise or may be applied together and/or sequentially with further active compounds. These further compounds can be fertilizers or micronutrient donors or other preparations that influence plant growth, such as inoculants.

The compositions according to the invention are effective against harmful microorganisms, such as microorganisms, that cause phytopathogenic diseases, in particular against phytopathogenic fungi and bacteria. The compositions according to the invention are effective especially against phytopathogenic fungi belonging to the following classes: Ascomycetes (e.g. Venturia, Podosphaera, Erysiphe, Monilinia, Mycosphaerella, Uncinula); Basidiomycetes (e.g. the genus Hemileia, Rhizoctonia, Phakopsora, Puccinia, Ustilago, Tilletia); Fungi imperfecti (also known as Deuteromycetes; e.g. Botrytis, Helminthosporium, Rhynchosporium, Fusarium, Zymoseptoria, Cercospora, Alternaria, Pyricularia and Pseudocercosporella); Oomycetes (e.g. Phytophthora, Peronospora, Pseudoperonospora, Albugo, Bremia, Pythium, Pseudosclerospora, Plasmopara).

The compositions according to the invention are furthermore particularly effective against seedborne and soilborne diseases, such as Alternaria spp., Ascochyta spp., Botrytis cinerea, Cercospora spp., Claviceps purpurea, Cochliobolus sativus, Colletotrichum spp., Epicoccum spp., Fusarium graminearum, Fusarium moniliforme, Fusarium oxysporum, Fusarium proliferatum, Fusarium solani, Fusarium subglutinans, Gäumannomyces graminis, Helminthosporium spp., Microdochium nivale, Phoma spp., Pyrenophora graminea, Pyricularia oryzae, Rhizoctonia solani, Rhizoctonia cerealis, Sclerotinia spp., Zymoseptoria spp., Sphacelotheca reilliana, Tilletia spp., Typhula incarnata, Urocystis occulta, Ustilago spp. or Verticillium spp.; in particular against pathogens of cereals, such as wheat, barley, rye or oats; maize; rice; cotton; soybean; turf; sugarbeet; oil seed rape; potatoes; pulse crops, such as peas, lentils or chickpea; and sunflower.

The compositions according to the invention are furthermore particularly effective against post-harvest diseases such as Botrytis cinerea, Colletotrichum musae, Curvularia lunata, Fusarium semitecum, Geotrichum candidum, Monilinia fructicola, Monilinia fructigena, Monilinia laxa, Mucor piriformis, Penicilium italicum, Penicilium solitum, Penicillium digitatum or Penicillium expansum in particular against pathogens of fruits, such as pomefruits, for example apples and pears, stone fruits, for example peaches and plums, citrus, melons, papaya, kiwi, mango, berries, for example strawberries, avocados, pomegranates and bananas, and nuts.

The compositions according to the invention are particularly useful for controlling the following diseases on the following crops: Alternaria species in fruit and vegetables and potato; Botrytis cinerea in strawberries, tomatoes, sunflower, pulse crops, vegetables and grapes; Rhizoctonia solani in potato and vegetables, Uncinula necator in grape, Cladosporium cucumerinum, Didymella bryoniae, Sphaerotheca fuliginea and Glomerella lagenarium in cucurbits, Leveillula taurica in cucurbits and solanacious crops, Fusarium spp in cereals, Leptosphaeria spp. in cereals, Septoria spp in cereals, like Zymoseptoria tritici, Septoria species in oilseed crops, like Septoria glycines, Cercospora species in tomato, legumes, penatus and oilseed crops, like Mycosphaerella arachidicola in peanuts, or Cercospora sojinae and Cercospora kikuchii in soybeans.

A synergistic effect exists whenever the action of an active ingredient combination is greater than the sum of the actions of the individual components. The action to be expected E for a given active ingredient combination obeys the so-called COLBY formula and can be calculated as follows (COLBY, S. R. “Calculating synergistic and antagonistic responses of herbicide combination”. Weeds, Vol. 15, pages 20-22 1967):
ppm=milligrams of active ingredient (a.i.) per liter
X=% action by first active ingredient using p ppm of the active ingredient
Y=% action by second active ingredient using q ppm of the active ingredient.

According to Colby, the expected (additive) action of active ingredients A+B using p+q ppm of active ingredient is

$$E = X + Y - \frac{X \cdot Y}{100}$$

If the action actually observed O is greater than the expected action E, then the action of the combination is super-additive, i.e. there is a synergistic effect. In mathematical terms, synergism corresponds to a positive value for the difference of (O-E). In the case of purely complementary addition of activities (expected activity), said difference (O-E) is zero. A negative value of said difference (O-E) signals a loss of activity compared to the expected activity.

Table 2 sets out certain preferred combinations for controlling or preventing damage to a plant. Table 2 lists example application rates, stating the application rate for component (A), the compound of formula (I), and each specified compound forming component (B). Application rates are given as grams per hectare. Table 2 also lists key pests against which the combinations are particularly effective and key crops for which the inventive combinations are particularly advantageous. Each combination of components (A) and (B) and each combination of pest and crop is individually exemplified by Table 2.

TABLE 2

| | Application | | |
|---|---|---|---|
| | rate: | | |
| | g(A)/ha/ | | |
| Combination | g(B)/ha | Pests | Crops |
| A-1/B-1 | 75/50 | Myzus persicae | soybean |
| | | Aphis gossypii | corn |
| | | Aphis spp. | cotton |
| | | Rhopalosiphum padi | rice |
| | | Sitobion spp. | vegetables |
| | | Bemisia tabaci | cereals |
| | | Bemisia spp. | pome fruits |
| | | Trialeurodes vaporarium | stone fruits |
| | | Nilaparvata lugens | citrus |
| | | Laodelphax striatellus | |
| | | Sogatella furcifera | |
| | | Spodoptera frugiperda | |
| | | Spodoptera spp. | |
| | | Helicoverpa armigera | |
| | | Heliothis spp. | |
| | | Chrysodeixis includens | |
| | | Scirpophaga incertulas | |
| | | Chilo spp. | |
| | | Plutella xylostella | |
| | | Tuta absoluta | |
| | | Cydia pomonella | |
| | | Leucinodes spp. | |
| | | Euschistus heros | |
| | | Nezara viridula | |
| | | Piezodorus spp. | |
| | | Dichelops spp. | |
| | | Frankliniella occidentalis | |
| | | Thrips tabaci | |
| | | Thrips palmi | |
| | | Thrips spp. | |
| | | Scirtothrips dorsalis | |
| | | Diabrotica virgifera | |
| | | virgifera | |
| | | Diabrotica spp. | |
| | | Agriotes spp. | |
| | | Limonius spp. | |
| | | Melanotus spp. | |
| | | wireworms | |
| A-1/B-2 | 75/50 | Myzus persicae | soybean |
| | | Aphis gossypii | corn |
| | | Aphis spp. | cotton |
| | | Rhopalosiphum padi | rice |
| | | Sitobion spp. | vegetables |
| | | Bemisia tabaci | cereals |
| | | Bemisia spp. | pome fruits |
| | | Trialeurodes vaporarium | stone fruits |
| | | Nilaparvata lugens | citrus |
| | | Laodelphax striatellus | |
| | | Sogatella furcifera | |
| | | Spodoptera frugiperda | |
| | | Spodoptera spp. | |
| | | Helicoverpa armigera | |
| | | Heliothis spp. | |
| | | Chrysodeixis includens | |
| | | Scirpophaga incertulas | |
| | | Chilo spp. | |
| | | Plutella xylostella | |
| | | Tuta absoluta | |
| | | Cydia pomonella | |
| | | Leucinodes spp. | |
| | | Euschistus heros | |
| | | Nezara viridula | |
| | | Piezodorus spp. | |
| | | Dichelops spp. | |
| | | Frankliniella occidentalis | |
| | | Thrips tabaci | |
| | | Thrips palmi | |
| | | Thrips spp. | |
| | | Scirtothrips dorsalis | |
| | | Diabrotica virgifera | |
| | | virgifera | |
| | | Diabrotica spp. | |
| | | Agriotes spp. | |
| | | Limonius spp. | |
| | | Melanotus spp. | |
| | | wireworms | |
| A-1/B-3 | 75/120 | Myzus persicae | soybean |
| | | Aphis gossypii | cotton |
| | | Aphis spp. | vegetables |
| | | Rhopalosiphum padi | pome fruits |
| | | Sitobion spp. | stone fruits |
| | | Bemisia tabaci | citrus |
| | | Bemisia spp. | cereals |
| | | Trialeurodes vaporarium | rice |
| | | Nilaparvata lugens | |
| | | Laodelphax striatellus | |
| | | Sogatella furcifera | |
| A-1/B-4 | 75/75 | Spodoptera frugiperda | rice |
| | | Spodoptera spp. | vegetables |
| | | Helicoverpa armigera | pome fruits |
| | | Heliothis spp. | stone fruits |
| | | Scirpophaga incertulas | citrus |
| | | Chilo spp. | soybean |
| | | Plutella xylostella | cotton |
| | | Tuta absoluta | cereals |
| | | Cydia pomonella | |
| | | Leucinodes spp. | |
| | | Nilaparvata lugens | |
| | | Laodelphax striatellus | |
| | | Sogatella furcifera | |
| | | Myzus persicae | |
| | | Aphis gossypii | |
| | | Aphis spp. | |
| | | Rhopalosiphum padi | |
| | | Sitobion spp. | |
| | | Bemisia tabaci | |
| | | Bemisia spp. | |
| | | Trialeurodes vaporarium | |
| A-1/B-5 | 75/75 | Spodoptera frugiperda | rice |
| | | Spodoptera spp. | vegetables |
| | | Helicoverpa armigera | pome fruits |
| | | Heliothis spp. | stone fruits |
| | | Scirpophaga incertulas | citrus |
| | | Chilo spp. | soybean |
| | | Plutella xylostella | cotton |
| | | Tuta absoluta | cereals |
| | | Cydia pomonella | |
| | | Leucinodes spp. | |
| | | Nilaparvata lugens | |
| | | Laodelphax striatellus | |
| | | Sogatella furcifera | |
| | | Myzus persicae | |
| | | Aphis gossypii | |
| | | Aphis spp. | |
| | | Rhopalosiphum padi | |
| | | Sitobion spp. | |
| | | Bemisia tabaci | |
| | | Bemisia spp. | |
| | | Trialeurodes vaporarium | |
| A-1/B-6 | 75/75 | Spodoptera frugiperda | rice |
| | | Spodoptera spp. | vegetables |
| | | Helicoverpa armigera | pome fruits |
| | | Heliothis spp. | stone fruits |
| | | Scirpophaga incertulas | citrus |
| | | Chilo spp. | soybean |
| | | Plutella xylostella | cotton |
| | | Tuta absoluta | cereals |
| | | Cydia pomonella | |
| | | Leucinodes spp. | |
| | | Nilaparvata lugens | |
| | | Laodelphax striatellus | |
| | | Sogatella furcifera | |
| | | Myzus persicae | |
| | | Aphis gossypii | |
| | | Aphis spp. | |
| | | Rhopalosiphum padi | |
| | | Sitobion spp. | |
| | | Bemisia tabaci | |
| | | Bemisia spp. | |
| | | Trialeurodes vaporarium | |
| A-1/B-7 | 75/75 | Spodoptera frugiperda | rice |
| | | Spodoptera spp. | vegetables |
| | | Helicoverpa armigera | pome fruits |
| | | Heliothis spp. | stone fruits |
| | | Scirpophaga incertulas | citrus |
| | | Chilo spp. | soybean |
| | | Plutella xylostella | cotton |
| | | Tuta absoluta | cereals |
| | | Cydia pomonella | |
| | | Leucinodes spp. | |
| | | Nilaparvata lugens | |
| | | Laodelphax striatellus | |
| | | Sogatella furcifera | |
| | | Myzus persicae | |
| | | Aphis gossypii | |
| | | Aphis spp. | |
| | | Rhopalosiphum padi | |
| | | Sitobion spp. | |
| | | Bemisia tabaci | |
| | | Bemisia spp. | |
| | | Trialeurodes vaporarium | |
| A-1/B-8 | 75/60 | Myzus persicae | soybean |
| | | Aphis gossypii | cotton |
| | | Aphis spp. | vegetables |
| | | Rhopalosiphum padi | pome fruits |
| | | Sitobion spp. | stone fruits |
| | | Bemisia tabaci | citrus |
| | | Bemisia spp. | cereals |
| | | Trialeurodes vaporarium | rice |
| | | Nilaparvata lugens | |
| | | Laodelphax striatellus | |
| | | Sogatella furcifera | |
| A-1/B-9 | 75/60 | Myzus persicae | soybean |
| | | Aphis gossypii | cotton |
| | | Aphis spp. | vegetables |
| | | Rhopalosiphum padi | pome fruits |
| | | Sitobion spp. | stone fruits |
| | | Bemisia tabaci | citrus |
| | | Bemisia spp. | cereals |
| | | Trialeurodes vaporarium | rice |
| | | Nilaparvata lugens | |
| | | Laodelphax striatellus | |
| | | Sogatella furcifera | |
| A-1/B-10 | 75/60 | Myzus persicae | soybean |
| | | Aphis gossypii | cotton |
| | | Aphis spp. | vegetables |
| | | Rhopalosiphum padi | pome fruits |
| | | Sitobion spp. | stone fruits |
| | | Bemisia tabaci | citrus |
| | | Bemisia spp. | cereals |
| | | Trialeurodes vaporarium | rice |
| | | Nilaparvata lugens | |
| | | Laodelphax striatellus | |
| | | Sogatella furcifera | |
| A-1/B-11 | 75/60 | Myzus persicae | soybean |
| | | Aphis gossypii | cotton |
| | | Aphis spp. | vegetables |
| | | Rhopalosiphum padi | pome fruits |
| | | Sitobion spp. | stone fruits |
| | | Bemisia tabaci | citrus |
| | | Bemisia spp. | cereals |
| | | Trialeurodes vaporarium | rice |
| | | Nilaparvata lugens | |
| | | Laodelphax striatellus | |
| | | Sogatella furcifera | |
| A-1/B-12 | 75/60 | Myzus persicae | soybean |
| | | Aphis gossypii | cotton |
| | | Aphis spp. | vegetables |
| | | Rhopalosiphum padi | pome fruits |
| | | Sitobion spp. | stone fruits |
| | | Bemisia tabaci | citrus |
| | | Bemisia spp. | cereals |
| | | Trialeurodes vaporarium | rice |
| | | Nilaparvata lugens | |
| | | Laodelphax striatellus | |
| | | Sogatella furcifera | |
| A-1/B-13 | 75/750 | Myzus persicae | soybean |
| | | Aphis gossypii | corn |
| | | Aphis spp. | cotton |
| | | Rhopalosiphum padi | rice |
| | | Sitobion spp. | vegetables |
| | | Bemisia tabaci | cereals |
| | | Bemisia spp. | pome fruits |
| | | Trialeurodes vaporarium | stone fruits |
| | | Nilaparvata lugens | citrus |
| | | Laodelphax striatellus | |
| | | Sogatella furcifera | |
| | | Spodoptera frugiperda | |
| | | Spodoptera spp. | |
| | | Helicoverpa armigera | |
| | | Heliothis spp. | |
| | | Chrysodeixis includens | |
| | | Scirpophaga incertulas | |
| | | Chilo spp. | |
| | | Plutella xylostella | |
| | | Tuta absoluta | |
| | | Cydia pomonella | |
| | | Leucinodes spp. | |
| | | Euschistus heros | |
| | | Nezara viridula | |
| | | Piezodorus spp. | |
| | | Dichelops spp. | |
| | | Frankliniella occidentalis | |
| | | Thrips tabaci | |
| | | Thrips palmi | |
| | | Thrips spp. | |
| | | Scirtothrips dorsalis | |
| | | Diabrotica virgifera | |
| | | virgifera | |
| | | Diabrotica spp. | |
| | | Agriotes spp. | |
| | | Limonius spp. | |
| | | Melanotus spp. | |
| | | wireworms | |
| A-1/B-31 | 75/50 | Myzus persicae | soybean |
| | | Aphis gossypii | corn |
| | | Aphis spp. | cotton |
| | | Rhopalosiphum padi | rice |
| | | Sitobion spp. | vegetables |
| | | Bemisia tabaci | cereals |
| | | Bemisia spp. | pome fruits |
| | | Trialeurodes vaporarium | stone fruits |
| | | Nilaparvata lugens | citrus |
| | | Laodelphax striatellus | |
| | | Sogatella furcifera | |
| | | Spodoptera frugiperda | |
| | | Spodoptera spp. | |
| | | Helicoverpa armigera | |
| | | Heliothis spp. | |
| | | Chrysodeixis includens | |
| | | Scirpophaga incertulas | |
| | | Chilo spp. | |
| | | Plutella xylostella | |
| | | Tuta absoluta | |
| | | Cydia pomonella | |
| | | Leucinodes spp. | |
| | | Euschistus heros | |
| | | Nezara viridula | |
| | | Piezodorus spp. | |
| | | Dichelops spp. | |
| | | Frankliniella occidentalis | |
| | | Thrips tabaci | |
| | | Thrips palmi | |
| | | Thrips spp. | |
| | | Scirtothrips dorsalis | |
| | | Diabrotica virgifera | |
| | | virgifera | |
| | | Diabrotica spp. | |
| | | Agriotes spp. | |
| | | Limonius spp. | |
| | | Melanotus spp. | |
| | | wireworms | |
| A-1/B-32 | 75/50 | Myzus persicae | soybean |
| | | Aphis gossypii | corn |
| | | Aphis spp. | cotton |
| | | Rhopalosiphum padi | rice |
| | | Sitobion spp. | vegetables |
| | | Bemisia tabaci | cereals |
| | | Bemisia spp. | pome fruits |
| | | Trialeurodes vaporarium | stone fruits |
| | | Nilaparvata lugens | citrus |
| | | Laodelphax striatellus | |
| | | Sogatella furcifera | |
| | | Spodoptera frugiperda | |
| | | Spodoptera spp. | |
| | | Helicoverpa armigera | |
| | | Heliothis spp. | |
| | | Chrysodeixis includens | |
| | | Scirpophaga incertulas | |
| | | Chilo spp. | |
| | | Plutella xylostella | |
| | | Tuta absoluta | |
| | | Cydia pomonella | |
| | | Leucinodes spp. | |
| | | Euschistus heros | |
| | | Nezara viridula | |
| | | Piezodorus spp. | |
| | | Dichelops spp. | |
| | | Frankliniella occidentalis | |
| | | Thrips tabaci | |
| | | Thrips palmi | |
| | | Thrips spp. | |
| | | Scirtothrips dorsalis | |
| | | Diabrotica virgifera | |
| | | virgifera | |
| | | Diabrotica spp. | |
| | | Agriotes spp. | |
| | | Limonius spp. | |
| | | Melanotus spp. | |
| | | wireworms | |
| A-1/B-38 | 75/75 | Myzus persicae | soybean |
| | | Aphis gossypii | cotton |
| | | Aphis spp. | vegetables |
| | | Rhopalosiphum padi | pome fruits |
| | | Sitobion spp. | stone fruits |
| | | Bemisia tabaci | citrus |
| | | Bemisia spp. | cereals |
| | | Trialeurodes vaporarium | rice |
| | | Nilaparvata lugens | |
| | | Laodelphax striatellus | |
| | | Sogatella furcifera | |
| A-1/B-39 | 75/80 | Myzus persicae | soybean |
| | | Aphis gossypii | cotton |
| | | Aphis spp. | vegetables |
| | | Rhopalosiphum padi | pome fruits |
| | | Sitobion spp. | stone fruits |
| | | Bemisia tabaci | citrus |
| | | Bemisia spp. | cereals |
| | | Trialeurodes vaporarium | rice |
| | | Nilaparvata lugens | |
| | | Laodelphax striatellus | |
| | | Sogatella furcifera | |
| A-1/B-40 | 75/50 | Myzus persicae | soybean |
| | | Aphis gossypii | corn |
| | | Aphis spp. | cotton |
| | | Rhopalosiphum padi | rice |
| | | Sitobion spp. | vegetables |
| | | Bemisia tabaci | cereals |
| | | Bemisia spp. | pome fruits |
| | | Trialeurodes vaporarium | stone fruits |
| | | Nilaparvata lugens | citrus |
| | | Laodelphax striatellus | |
| | | Sogatella furcifera | |
| | | Spodoptera frugiperda | |
| | | Spodoptera spp. | |
| | | Helicoverpa armigera | |
| | | Heliothis spp. | |
| | | Chrysodeixis includens | |
| | | Scirpophaga incertulas | |
| | | Chilo spp. | |
| | | Plutella xylostella | |
| | | Tuta absoluta | |
| | | Cydia pomonella | |
| | | Leucinodes spp. | |
| | | Euschistus heros | |
| | | Nezara viridula | |
| | | Piezodorus spp. | |
| | | Dichelops spp. | |
| | | Frankliniella occidentalis | |
| | | Thrips tabaci | |
| | | Thrips palmi | |
| | | Thrips spp. | |
| | | Scirtothrips dorsalis | |
| | | Diabrotica virgifera | |
| | | virgifera | |
| | | Diabrotica spp. | |
| | | Agriotes spp. | |
| | | Limonius spp. | |
| | | Melanotus spp. | |
| | | wireworms | |
| A-1/B-41 | 75/120 | Myzus persicae | soybean |
| | | Aphis gossypii | cotton |
| | | Aphis spp. | vegetables |
| | | Rhopalosiphum padi | pome fruits |
| | | Sitobion spp. | stone fruits |
| | | Bemisia tabaci | citrus |
| | | Bemisia spp. | cereals |
| | | Trialeurodes vaporarium | rice |
| | | Nilaparvata lugens | |
| | | Laodelphax striatellus | |
| | | Sogatella furcifera | |
| A-1/B-42 | 75/30 | Myzus persicae | soybean |
| | | Aphis gossypii | cotton |
| | | Aphis spp. | vegetables |
| | | Rhopalosiphum padi | pome fruits |
| | | Sitobion spp. | stone fruits |
| | | Bemisia tabaci | citrus |
| | | Bemisia spp. | cereals |
| | | Trialeurodes vaporarium | rice |
| | | Nilaparvata lugens | |
| | | Laodelphax striatellus | |
| | | Sogatella furcifera | |
| A-1/B-43 | 75/50 | Myzus persicae | soybean |
| | | Aphis gossypii | corn |
| | | Aphis spp. | cotton |
| | | Rhopalosiphum padi | rice |
| | | Sitobion spp. | vegetables |
| | | Bemisia tabaci | cereals |
| | | Bemisia spp. | pome fruits |
| | | Trialeurodes vaporarium | stone fruits |
| | | Nilaparvata lugens | citrus |
| | | Laodelphax striatellus | |
| | | Sogatella furcifera | |
| | | Spodoptera frugiperda | |
| | | Spodoptera spp. | |
| | | Helicoverpa armigera | |
| | | Heliothis spp. | |
| | | Chrysodeixis includens | |
| | | Scirpophaga incertulas | |
| | | Chilo spp. | |
| | | Plutella xylostella | |
| | | Tuta absoluta | |
| | | Cydia pomonella | |
| | | Leucinodes spp. | |
| | | Euschistus heros | |
| | | Nezara viridula | |
| | | Piezodorus spp. | |
| | | Dichelops spp. | |
| | | Frankliniella occidentalis | |
| | | Thrips tabaci | |
| | | Thrips palmi | |
| | | Thrips spp. | |
| | | Scirtothrips dorsalis | |
| | | Diabrotica virgifera | |
| | | virgifera | |
| | | Diabrotica spp. | |
| | | Agriotes spp. | |
| | | Limonius spp. | |
| | | Melanotus spp. | |
| | | wireworms | |
| A-1/B-44 | 75/50 | Myzus persicae | soybean |
| | | Aphis gossypii | corn |
| | | Aphis spp. | cotton |
| | | Rhopalosiphum padi | rice |
| | | Sitobion spp. | vegetables |
| | | Bemisia tabaci | cereals |
| | | Bemisia spp. | pome fruits |
| | | Trialeurodes vaporarium | stone fruits |
| | | Nilaparvata lugens | citrus |
| | | Laodelphax striatellus | |
| | | Sogatella furcifera | |
| | | Spodoptera frugiperda | |
| | | Spodoptera spp. | |
| | | Helicoverpa armigera | |
| | | Heliothis spp. | |
| | | Chrysodeixis includens | |
| | | Scirpophaga incertulas | |
| | | Chilo spp. | |
| | | Plutella xylostella | |
| | | Tuta absoluta | |
| | | Cydia pomonella | |
| | | Leucinodes spp. | |
| | | Euschistus heros | |
| | | Nezara viridula | |
| | | Piezodorus spp. | |
| | | Dichelops spp. | |
| | | Frankliniella occidentalis | |
| | | Thrips tabaci | |
| | | Thrips palmi | |
| | | Thrips spp. | |
| | | Scirtothrips dorsalis | |
| | | Diabrotica virgifera | |
| | | virgifera | |
| | | Diabrotica spp. | |
| | | Agriotes spp. | |
| | | Limonius spp. | |
| | | Melanotus spp. | |
| | | wireworms | |
| A-1/B-45 | 75/75 | Myzus persicae | soybean |
| | | Aphis gossypii | corn |
| | | Aphis spp. | cotton |
| | | Rhopalosiphum padi | rice |
| | | Sitobion spp. | vegetables |
| | | Bemisia tabaci | cereals |
| | | Bemisia spp. | pome fruits |
| | | Trialeurodes vaporarium | stone fruits |
| | | Nilaparvata lugens | citrus |
| | | Laodelphax striatellus | |
| | | Sogatella furcifera | |
| | | Spodoptera frugiperda | |
| | | Spodoptera spp. | |
| | | Helicoverpa armigera | |
| | | Heliothis spp. | |
| | | Chrysodeixis includens | |
| | | Scirpophaga incertulas | |
| | | Chilo spp. | |
| | | Plutella xylostella | |
| | | Tuta absoluta | |
| | | Cydia pomonella | |
| | | Leucinodes spp. | |
| | | Euschistus heros | |
| | | Nezara viridula | |
| | | Piezodorus spp. | |
| | | Dichelops spp. | |
| | | Frankliniella occidentalis | |
| | | Thrips tabaci | |
| | | Thrips palmi | |
| | | Thrips spp. | |
| | | Scirtothrips dorsalis | |
| A-1/B-46 | 75/30 | Myzus persicae | soybean |
| | | Aphis gossypii | corn |
| | | Aphis spp. | cotton |
| | | Rhopalosiphum padi | vegetables |
| | | Sitobion spp. | pome fruits |
| | | Bemisia tabaci | stone fruits |
| | | Bemisia spp. | citrus |
| | | Trialeurodes vaporarium | cereals |
| | | Nilaparvata lugens | rice |
| | | Laodelphax striatellus | |
| | | Sogatella furcifera | |
| | | Euschistus heros | |
| | | Nezara viridula | |
| | | Piezodorus spp. | |
| | | Dichelops spp. | |
| | | Frankliniella occidentalis | |
| | | Thrips tabaci | |
| | | Thrips palmi | |
| | | Thrips spp. | |
| | | Scirtothrips dorsalis | |
| | | Diabrotica virgifera | |
| | | virgifera | |
| | | Diabrotica spp. | |
| | | Agriotes spp. | |
| | | Limonius spp. | |
| | | Melanotus spp. | |
| | | wireworms | |
| A-1/B-47 | 75/150 | Myzus persicae | soybean |
| | | Aphis gossypii | corn |
| | | Aphis spp. | cotton |
| | | Rhopalosiphum padi | vegetables |
| | | Sitobion spp. | pome fruits |
| | | Bemisia | stone fruits |
| | | tabaci | citrus |
| | | Bemisia spp. | cereals |
| | | Trialeurodes vaporarium | rice |
| | | Nilaparvata lugens | |
| | | Laodelphax striatellus | |
| | | Sogatella furcifera | |
| | | Euschistus heros | |
| | | Nezara viridula | |
| | | Piezodorus spp. | |
| | | Dichelops spp. | |
| | | Frankliniella occidentalis | |
| | | Thrips tabaci | |
| | | Thrips palmi | |
| | | Thrips spp. | |
| | | Scirtothrips dorsalis | |
| | | Diabrotica virgifera | |
| | | virgifera | |
| | | Diabrotica spp. | |
| | | Agriotes spp. | |
| | | Limonius spp. | |
| | | Melanotus spp. | |
| | | wireworms | |
| A-1/B-48 | 75/30 | Myzus persicae | soybean |
| | | Aphis gossypii | corn |
| | | Aphis spp. | cotton |
| | | Rhopalosiphum padi | vegetables |
| | | Sitobion spp. | pome fruits |
| | | Bemisia tabaci | stone fruits |
| | | Bemisia spp. | citrus |
| | | Trialeurodes vaporarium | cereals |
| | | Nilaparvata lugens | rice |
| | | Laodelphax striatellus | |
| | | Sogatella furcifera | |
| | | Euschistus heros | |
| | | Nezara viridula | |
| | | Piezodorus spp. | |
| | | Dichelops spp. | |
| | | Frankliniella occidentalis | |
| | | Thrips tabaci | |
| | | Thrips palmi | |
| | | Thrips spp. | |
| | | Scirtothrips dorsalis | |
| | | Diabrotica virgifera | |
| | | virgifera | |
| | | Diabrotica spp. | |
| | | Agriotes spp. | |
| | | Limonius spp. | |
| | | Melanotus spp. | |
| | | wireworms | |
| A-1/B-49 | 75/30 | Myzus persicae | soybean |
| | | Aphis gossypii | corn |
| | | Aphis spp. | cotton |
| | | Rhopalosiphum padi | vegetables |
| | | Sitobion spp. | pome fruits |
| | | Bemisia tabaci | stone fruits |
| | | Bemisia spp. | citrus |
| | | Trialeurodes vaporarium | cereals |
| | | Nilaparvata lugens | rice |
| | | Laodelphax striatellus | |
| | | Sogatella furcifera | |
| | | Euschistus heros | |
| | | Nezara viridula | |
| | | Piezodorus spp. | |
| | | Dichelops spp. | |
| | | Frankliniella occidentalis | |
| | | Thrips tabaci | |
| | | Thrips palmi | |
| | | Thrips spp. | |
| | | Scirtothrips dorsalis | |
| | | Diabrotica virgifera | |
| | | virgifera | |
| | | Diabrotica spp. | |
| | | Agriotes spp. | |
| | | Limonius spp. | |
| | | Melanotus spp. | |
| | | wireworms | |
| A-1/B-50 | 75/30 | Myzus persicae | soybean |
| | | Aphis gossypii | corn |
| | | Aphis spp. | cotton |
| | | Rhopalosiphum padi | vegetables |
| | | Sitobion spp. | pome fruits |
| | | Bemisia tabaci | stone fruits |
| | | Bemisia spp. | citrus |
| | | Trialeurodes vaporarium | cereals |
| | | Nilaparvata lugens | rice |
| | | Laodelphax striatellus | |
| | | Sogatella furcifera | |
| | | Euschistus heros | |
| | | Nezara viridula | |
| | | Piezodorus spp. | |
| | | Dichelops spp. | |
| | | Frankliniella occidentalis | |
| | | Thrips tabaci | |
| | | Thrips palmi | |
| | | Thrips spp. | |
| | | Scirtothrips dorsalis | |
| | | Diabrotica virgifera | |
| | | virgifera | |
| | | Diabrotica spp. | |
| | | Agriotes spp. | |
| | | Limonius spp. | |
| | | Melanotus spp. | |
| | | wireworms | |
| A-1/B-51 | 75/30 | Myzus persicae | soybean |
| | | Aphis gossypii | corn |
| | | Aphis spp. | cotton |
| | | Rhopalosiphum padi | vegetables |
| | | Sitobion spp. | pome fruits |
| | | Bemisia tabaci | stone fruits |
| | | Bemisia spp. | citrus |
| | | Trialeurodes vaporarium | cereals |
| | | Nilaparvata lugens | rice |
| | | Laodelphax striatellus | |
| | | Sogatella furcifera | |
| | | Euschistus heros | |
| | | Nezara viridula | |
| | | Piezodorus spp. | |
| | | Dichelops spp. | |
| | | Frankliniella occidentalis | |
| | | Thrips tabaci | |
| | | Thrips palmi | |
| | | Thrips spp. | |
| | | Scirtothrips dorsalis | |
| | | Diabrotica virgifera | |
| | | virgifera | |
| | | Diabrotica spp. | |
| | | Agriotes spp. | |
| | | Limonius spp. | |
| | | Melanotus spp. | |
| | | wireworms | |
| A-1/B-52 | 75/30 | Myzus persicae | soybean |
| | | Aphis gossypii | corn |
| | | Aphis spp. | cotton |
| | | Rhopalosiphum padi | vegetables |
| | | Sitobion spp. | pome fruits |
| | | Bemisia tabaci | stone fruits |
| | | Bemisia spp. | citrus |
| | | Trialeurodes vaporarium | cereals |
| | | Nilaparvata lugens | rice |
| | | Laodelphax striatellus | |
| | | Sogatella furcifera | |
| | | Euschistus heros | |
| | | Nezara viridula | |
| | | Piezodorus spp. | |
| | | Dichelops spp. | |
| | | Frankliniella occidentalis | |
| | | Thrips tabaci | |
| | | Thrips palmi | |
| | | Thrips spp. | |
| | | Scirtothrips dorsalis | |
| | | Diabrotica virgifera | |
| | | virgifera | |
| | | Diabrotica spp. | |
| | | Agriotes spp. | |
| | | Limonius spp. | |
| | | Melanotus spp. | |
| | | wireworms | |
| A-1/B-53 | 75/30 | Myzus persicae | soybean |
| | | Aphis gossypii | corn |
| | | Aphis spp. | cotton |
| | | Rhopalosiphum padi | vegetables |
| | | Sitobion spp. | pome fruits |
| | | Bemisia tabaci | stone fruits |
| | | Bemisia spp. | citrus |
| | | Trialeurodes vaporarium | cereals |
| | | Nilaparvata lugens | rice |
| | | Laodelphax striatellus | |
| | | Sogatella furcifera | |
| | | Euschistus heros | |
| | | Nezara viridula | |
| | | Piezodorus spp. | |
| | | Dichelops spp. | |
| | | Frankliniella occidentalis | |
| | | Thrips tabaci | |
| | | Thrips palmi | |
| | | Thrips spp. | |
| | | Scirtothrips dorsalis | |
| | | Diabrotica virgifera | |
| | | virgifera | |
| | | Diabrotica spp. | |
| | | Agriotes spp. | |
| | | Limonius spp. | |
| | | Melanotus spp. | |
| | | wireworms | |
| A-1/B-54 | 75/30 | Myzus persicae | soybean |
| | | Aphis gossypii | corn |
| | | Aphis spp. | cotton |
| | | Rhopalosiphum padi | rice |
| | | Sitobion spp. | vegetables |
| | | Bemisia tabaci | cereals |
| | | Bemisia spp. | pome fruits |
| | | Trialeurodes vaporarium | stone fruits |
| | | Nilaparvata lugens | citrus |
| | | Laodelphax striatellus | |
| | | Sogatella furcifera | |
| | | Spodoptera frugiperda | |
| | | Spodoptera spp. | |
| | | Helicoverpa armigera | |
| | | Heliothis spp. | |
| | | Chrysodeixis includens | |
| | | Scirpophaga incertulas | |
| | | Chilo spp. | |
| | | Plutella xylostella | |
| | | Tuta absoluta | |
| | | Cydia pomonella | |
| | | Leucinodes spp. | |
| | | Euschistus heros | |
| | | Nezara viridula | |
| | | Piezodorus spp. | |
| | | Dichelops spp. | |
| | | Frankliniella occidentalis | |
| | | Thrips tabaci | |
| | | Thrips palmi | |
| | | Thrips spp. | |
| | | Scirtothrips dorsalis | |
| | | Diabrotica virgifera | |
| | | virgifera | |
| | | Diabrotica spp. | |
| | | Agriotes spp. | |
| | | Limonius spp. | |
| | | Melanotus spp. | |
| | | wireworms | |
| A-1/B-55 | 75/30 | Myzus persicae | soybean |
| | | Aphis gossypii | corn |
| | | Aphis spp. | cotton |
| | | Rhopalosiphum padi | rice |
| | | Sitobion spp. | vegetables |
| | | Bemisia tabaci | cereals |
| | | Bemisia spp. | pome fruits |
| | | Trialeurodes vaporarium | stone fruits |
| | | Nilaparvata lugens | citrus |
| | | Laodelphax striatellus | |
| | | Sogatella furcifera | |
| | | Spodoptera frugiperda | |
| | | Spodoptera spp. | |
| | | Helicoverpa armigera | |
| | | Heliothis spp. | |
| | | Chrysodeixis includens | |
| | | Scirpophaga incertulas | |
| | | Chilo spp. | |
| | | Plutella xylostella | |
| | | Tuta absoluta | |
| | | Cydia pomonella | |
| | | Leucinodes spp. | |
| | | Euschistus heros | |
| | | Nezara viridula | |
| | | Piezodorus spp. | |
| | | Dichelops spp. | |
| | | Frankliniella occidentalis | |
| | | Thrips tabaci | |
| | | Thrips palmi | |
| | | Thrips spp. | |
| | | Scirtothrips dorsalis | |
| | | Diabrotica virgifera | |
| | | virgifera | |
| | | Diabrotica spp. | |
| | | Agriotes spp. | |
| | | Limonius spp. | |
| | | Melanotus spp. | |
| | | wireworms | |
| A-1/B-56 | 75/30 | Myzus persicae | soybean |
| | | Aphis gossypii | corn |
| | | Aphis spp. | cotton |
| | | Rhopalosiphum padi | rice |
| | | Sitobion spp. | vegetables |
| | | Bemisia tabaci | cereals |
| | | Bemisia spp. | pome fruits |
| | | Trialeurodes vaporarium | stone fruits |
| | | Nilaparvata lugens | citrus |
| | | Laodelphax striatellus | |
| | | Sogatella furcifera | |
| | | Spodoptera frugiperda | |
| | | Spodoptera spp. | |
| | | Helicoverpa armigera | |
| | | Heliothis spp. | |
| | | Chrysodeixis includens | |
| | | Scirpophaga incertulas | |
| | | Chilo spp. | |
| | | Plutella xylostella | |
| | | Tuta absoluta | |
| | | Cydia pomonella | |
| | | Leucinodes spp. | |
| | | Euschistus heros | |
| | | Nezara viridula | |
| | | Piezodorus spp. | |
| | | Dichelops spp. | |
| | | Frankliniella occidentalis | |
| | | Thrips tabaci | |
| | | Thrips palmi | |
| | | Thrips spp. | |
| | | Scirtothrips dorsalis | |
| | | Diabrotica virgifera | |
| | | virgifera | |
| | | Diabrotica spp. | |
| | | Agriotes spp. | |
| | | Limonius spp. | |
| | | Melanotus spp. | |
| | | wireworms | |
| A-1/B-57 | 75/30 | Myzus persicae | soybean |
| | | Aphis gossypii | corn |
| | | Aphis spp. | cotton |
| | | Rhopalosiphum padi | rice |
| | | Sitobion spp. | vegetables |
| | | Bemisia tabaci | cereals |
| | | Bemisia spp. | pome fruits |
| | | Trialeurodes vaporarium | stone fruits |
| | | Nilaparvata lugens | citrus |
| | | Laodelphax striatellus | |
| | | Sogatella furcifera | |
| | | Spodoptera frugiperda | |
| | | Spodoptera spp. | |
| | | Helicoverpa armigera | |
| | | Heliothis spp. | |
| | | Chrysodeixis includens | |
| | | Scirpophaga incertulas | |
| | | Chilo spp. | |
| | | Plutella xylostella | |
| | | Tuta absoluta | |
| | | Cydia pomonella | |
| | | Leucinodes spp. | |
| | | Euschistus heros | |
| | | Nezara viridula | |
| | | Piezodorus spp. | |
| | | Dichelops spp. | |
| | | Frankliniella occidentalis | |
| | | Thrips tabaci | |
| | | Thrips palmi | |
| | | Thrips spp. | |
| | | Scirtothrips dorsalis | |
| | | Diabrotica virgifera | |
| | | virgifera | |
| | | Diabrotica spp. | |
| | | Agriotes spp. | |
| | | Limonius spp. | |
| | | Melanotus spp. | |
| | | wireworms | |
| A-1/B-58 | 75/60 | Myzus persicae | soybean |
| | | Aphis gossypii | cotton |
| | | Aphis spp. | vegetables |
| | | Rhopalosiphum padi | pome fruits |
| | | Sitobion spp. | stone fruits |
| | | Bemisia tabaci | citrus |
| | | Bemisia spp. | cereals |
| | | Trialeurodes vaporarium | rice |
| | | Nilaparvata lugens | |
| | | Laodelphax striatellus | |
| | | Sogatella furcifera | |
| A-1/B-59 | 75/100 | Myzus persicae | soybean |
| | | Aphis gossypii | corn |
| | | Aphis spp. | cotton |
| | | Rhopalosiphum padi | rice |
| | | Sitobion spp. | vegetables |
| | | Bemisia tabaci | cereals |
| | | Bemisia spp. | pome fruits |
| | | Trialeurodes vaporarium | stone fruits |
| | | Nilaparvata lugens | citrus |
| | | Laodelphax striatellus | |
| | | Sogatella furcifera | |
| | | Spodoptera frugiperda | |
| | | Spodoptera spp. | |
| | | Helicoverpa armigera | |
| | | Heliothis spp. | |
| | | Chrysodeixis includens | |
| | | Scirpophaga incertulas | |
| | | Chilo spp. | |
| | | Plutella xylostella | |
| | | Tuta absoluta | |
| | | Cydia pomonella | |
| | | Leucinodes spp. | |
| | | Euschistus heros | |
| | | Nezara viridula | |
| | | Piezodorus spp. | |
| | | Dichelops spp. | |
| A-1/B-60 | 75/30 | Myzus persicae | soybean |
| | | Aphis gossypii | corn |
| | | Aphis spp. | cotton |
| | | Rhopalosiphum padi | rice |
| | | Sitobion spp. | vegetables |
| | | Bemisia tabaci | cereals |
| | | Bemisia spp. | pome fruits |
| | | Trialeurodes vaporarium | stone fruits |
| | | Nilaparvata lugens | citrus |
| | | Laodelphax striatellus | |
| | | Sogatella furcifera | |
| | | Spodoptera frugiperda | |
| | | Spodoptera spp. | |
| | | Helicoverpa armigera | |
| | | Heliothis spp. | |
| | | Chrysodeixis includens | |
| | | Scirpophaga incertulas | |
| | | Chilo spp. | |
| | | Plutella xylostella | |
| | | Tuta absoluta | |
| | | Cydia pomonella | |
| | | Leucinodes spp. | |
| | | Euschistus heros | |
| | | Nezara viridula | |
| | | Piezodorus spp. | |
| | | Dichelops spp. | |
| | | Frankliniella occidentalis | |
| | | Thrips tabaci | |
| | | Thrips palmi | |
| | | Thrips spp. | |
| | | Scirtothrips dorsalis | |
| | | Diabrotica virgifera | |
| | | virgifera | |
| | | Diabrotica spp. | |
| | | Agriotes spp. | |
| | | Limonius spp. | |
| | | Melanotus spp. | |
| | | wireworms | |
| A-1/B-61 | 75/30 | Myzus persicae | soybean |
| | | Aphis gossypii | corn |
| | | Aphis spp. | cotton |
| | | Rhopalosiphum padi | rice |
| | | Sitobion spp. | vegetables |
| | | Bemisia tabaci | cereals |
| | | Bemisia spp. | pome fruits |
| | | Trialeurodes vaporarium | stone fruits |
| | | Nilaparvata lugens | citrus |
| | | Laodelphax striatellus | |
| | | Sogatella furcifera | |
| | | Spodoptera frugiperda | |
| | | Spodoptera spp. | |
| | | Helicoverpa armigera | |
| | | Heliothis spp. | |
| | | Chrysodeixis includens | |
| | | Scirpophaga incertulas | |
| | | Chilo spp. | |
| | | Plutella xylostella | |
| | | Tuta absoluta | |
| | | Cydia pomonella | |
| | | Leucinodes spp. | |
| | | Euschistus heros | |
| | | Nezara viridula | |
| | | Piezodorus spp. | |
| | | Dichelops spp. | |
| | | Frankliniella occidentalis | |
| | | Thrips tabaci | |
| | | Thrips palmi | |
| | | Thrips spp. | |
| | | Scirtothrips dorsalis | |
| | | Diabrotica virgifera | |
| | | virgifera | |
| | | Diabrotica spp. | |
| | | Agriotes spp. | |
| | | Limonius spp. | |
| | | Melanotus spp. | |
| | | wireworms | |
| A-1/B-62 | 75/30 | Myzus persicae | soybean |
| | | Aphis gossypii | corn |
| | | Aphis spp. | cotton |
| | | Rhopalosiphum padi | vegetables |
| | | Sitobion spp. | pome fruits |
| | | Bemisia tabaci | stone fruits |
| | | Bemisia spp. | citrus |
| | | Trialeurodes vaporarium | cereals |
| | | Nilaparvata lugens | rice |
| | | Laodelphax striatellus | |
| | | Sogatella furcifera | |
| | | Euschistus heros | |
| | | Nezara viridula | |
| | | Piezodorus spp. | |
| | | Dichelops spp. | |
| | | Frankliniella occidentalis | |
| | | Thrips tabaci | |
| | | Thrips palmi | |
| | | Thrips spp. | |
| | | Scirtothrips dorsalis | |
| | | Diabrotica virgifera | |
| | | virgifera | |
| | | Diabrotica spp. | |
| | | Agriotes spp. | |
| | | Limonius spp. | |
| | | Melanotus spp. | |
| | | wireworms | |
| | | Tetranychus urticae | |
| | | Tetranychus spp. | |
| | | Panonychus ulmi | |
| | | Panonychus citri | |
| | | Polyphagotarsonemus | |
| | | spp. | |
| | | Phyllocoptruta spp. | |
| | | Aculus spp. | |
| | | Brevipalpus spp. | |
| A-1/B-63 | 75/30 | Myzus persicae | soybean |
| | | Aphis gossypii | corn |
| | | Aphis spp. | cotton |
| | | Rhopalosiphum padi | rice |
| | | Sitobion spp. | vegetables |
| | | Bemisia tabaci | cereals |
| | | Bemisia spp. | pome fruits |
| | | Trialeurodes vaporarium | stone fruits |
| | | Nilaparvata lugens | citrus |
| | | Laodelphax striatellus | |
| | | Sogatella furcifera | |
| | | Spodoptera frugiperda | |
| | | Spodoptera spp. | |
| | | Helicoverpa armigera | |
| | | Heliothis spp. | |
| | | Chrysodeixis includens | |
| | | Scirpophaga incertulas | |
| | | Chilo spp. | |
| | | Plutella xylostella | |
| | | Tuta absoluta | |
| | | Cydia pomonella | |
| | | Leucinodes spp. | |
| | | Euschistus heros | |
| | | Nezara viridula | |
| | | Piezodorus spp. | |
| | | Dichelops spp. | |
| | | Frankliniella occidentalis | |
| | | Thrips tabaci | |
| | | Thrips | |
| | | palmi | |
| | | Thrips spp. | |
| | | Scirtothrips dorsalis | |
| | | Diabrotica virgifera | |
| | | virgifera | |
| | | Diabrotica spp. | |
| | | Agriotes spp. | |
| | | Limonius spp. | |
| | | Melanotus spp. | |
| | | wireworms | |
| A-1/B-64 | 75/30 | Myzus persicae | soybean |
| | | Aphis gossypii | corn |
| | | Aphis spp. | cotton |
| | | Rhopalosiphum padi | rice |
| | | Sitobion spp. | vegetables |
| | | Bemisia tabaci | cereals |
| | | Bemisia spp. | pome fruits |
| | | Trialeurodes vaporarium | stone fruits |
| | | Nilaparvata lugens | citrus |
| | | Laodelphax striatellus | |
| | | Sogatella furcifera | |
| | | Spodoptera frugiperda | |
| | | Spodoptera spp. | |
| | | Helicoverpa armigera | |
| | | Heliothis spp. | |
| | | Chrysodeixis includens | |
| | | Scirpophaga incertulas | |
| | | Chilo spp. | |
| | | Plutella xylostella | |
| | | Tuta absoluta | |
| | | Cydia pomonella | |
| | | Leucinodes spp. | |
| | | Euschistus heros | |
| | | Nezara viridula | |
| | | Piezodorus spp. | |
| | | Dichelops spp. | |
| | | Frankliniella occidentalis | |
| | | Thrips tabaci | |
| | | Thrips palmi | |
| | | Thrips spp. | |
| | | Scirtothrips dorsalis | |
| | | Diabrotica virgifera | |
| | | virgifera | |
| | | Diabrotica spp. | |
| | | Agriotes spp. | |
| | | Limonius spp. | |
| | | Melanotus spp. | |
| | | wireworms | |
| A-1/B-65 | 75/30 | Myzus persicae | soybean |
| | | Aphis gossypii | corn |
| | | Aphis spp. | cotton |
| | | Rhopalosiphum padi | rice |
| | | Sitobion spp. | vegetables |
| | | Bemisia tabaci | cereals |
| | | Bemisia spp. | pome fruits |
| | | Trialeurodes vaporarium | stone fruits |
| | | Nilaparvata lugens | citrus |
| | | Laodelphax striatellus | |
| | | Sogatella furcifera | |
| | | Spodoptera frugiperda | |
| | | Spodoptera spp. | |
| | | Helicoverpa armigera | |
| | | Heliothis spp. | |
| | | Chrysodeixis includens | |
| | | Scirpophaga incertulas | |
| | | Chilo spp. | |
| | | Plutella xylostella | |
| | | Tuta absoluta | |
| | | Cydia pomonella | |
| | | Leucinodes spp. | |
| | | Euschistus heros | |
| | | Nezara viridula | |
| | | Piezodorus spp. | |
| | | Dichelops spp. | |
| | | Frankliniella occidentalis | |
| | | Thrips tabaci | |
| | | Thrips palmi | |
| | | Thrips spp. | |
| | | Scirtothrips dorsalis | |
| | | Diabrotica virgifera | |
| | | virgifera | |
| | | Diabrotica spp. | |
| | | Agriotes spp. | |
| | | Limonius spp. | |
| | | Melanotus spp. | |
| | | wireworms | |
| A-1/B-66 | 75/30 | Myzus persicae | soybean |
| | | Aphis gossypii | corn |
| | | Aphis spp. | cotton |
| | | Rhopalosiphum padi | rice |
| | | Sitobion spp. | vegetables |
| | | Bemisia tabaci | cereals |
| | | Bemisia spp. | pome fruits |
| | | Trialeurodes vaporarium | stone fruits |
| | | Nilaparvata lugens | citrus |
| | | Laodelphax striatellus | |
| | | Sogatella furcifera | |
| | | Spodoptera frugiperda | |
| | | Spodoptera spp. | |
| | | Helicoverpa armigera | |
| | | Heliothis spp. | |
| | | Chrysodeixis includens | |
| | | Scirpophaga incertulas | |
| | | Chilo spp. | |
| | | Plutella xylostella | |
| | | Tuta absoluta | |
| | | Cydia pomonella | |
| | | Leucinodes spp. | |
| | | Euschistus heros | |
| | | Nezara viridula | |
| | | Piezodorus spp. | |
| | | Dichelops spp. | |
| | | Frankliniella occidentalis | |
| | | Thrips tabaci | |
| | | Thrips palmi | |
| | | Thrips spp. | |
| | | Scirtothrips dorsalis | |
| | | Diabrotica virgifera | |
| | | virgifera | |
| | | Diabrotica spp. | |
| | | Agriotes spp. | |
| | | Limonius spp. | |
| | | Melanotus spp. | |
| | | wireworms | |
| A-1/B-67 | 75/30 | Myzus persicae | soybean |
| | | Aphis gossypii | corn |
| | | Aphis spp. | cotton |
| | | Rhopalosiphum padi | rice |
| | | Sitobion spp. | vegetables |
| | | Bemisia tabaci | cereals |
| | | Bemisia spp. | pome fruits |
| | | Trialeurodes vaporarium | stone fruits |
| | | Nilaparvata lugens | citrus |
| | | Laodelphax striatellus | |
| | | Sogatella furcifera | |
| | | Spodoptera frugiperda | |
| | | Spodoptera spp. | |
| | | Helicoverpa armigera | |
| | | Heliothis spp. | |
| | | Chrysodeixis includens | |
| | | Scirpophaga incertulas | |
| | | Chilo spp. | |
| | | Plutella xylostella | |
| | | Tuta absoluta | |
| | | Cydia pomonella | |
| | | Leucinodes spp. | |
| | | Euschistus heros | |
| | | Nezara viridula | |
| | | Piezodorus spp. | |
| | | Dichelops spp. | |
| | | Frankliniella occidentalis | |
| | | Thrips tabaci | |
| | | Thrips palmi | |
| | | Thrips spp. | |
| | | Scirtothrips dorsalis | |
| | | Diabrotica virgifera | |
| | | virgifera | |
| | | Diabrotica spp. | |
| | | Agriotes spp. | |
| | | Limonius spp. | |
| | | Melanotus spp. | |
| | | wireworms | |
| A-1/B-68 | 75/30 | Myzus persicae | soybean |
| | | Aphis gossypii | corn |
| | | Aphis spp. | cotton |
| | | Rhopalosiphum padi | rice |
| | | Sitobion spp. | vegetables |
| | | Bemisia tabaci | cereals |
| | | Bemisia spp. | pome fruits |
| | | Trialeurodes vaporarium | stone fruits |
| | | Nilaparvata lugens | citrus |
| | | Laodelphax striatellus | |
| | | Sogatella furcifera | |
| | | Spodoptera frugiperda | |
| | | Spodoptera spp. | |
| | | Helicoverpa armigera | |
| | | Heliothis spp. | |
| | | Chrysodeixis includens | |
| | | Scirpophaga incertulas | |
| | | Chilo spp. | |
| | | Plutella xylostella | |
| | | Tuta absoluta | |
| | | Cydia pomonella | |
| | | Leucinodes spp. | |
| | | Euschistus heros | |
| | | Nezara viridula | |
| | | Piezodorus spp. | |
| | | Dichelops spp. | |
| | | Frankliniella occidentalis | |
| | | Thrips tabaci | |
| | | Thrips palmi | |
| | | Thrips spp. | |
| | | Scirtothrips dorsalis | |
| | | Diabrotica virgifera | |
| | | virgifera | |
| | | Diabrotica spp. | |
| | | Agriotes spp. | |
| | | Limonius spp. | |
| | | Melanotus spp. | |
| | | wireworms | |
| A-1/B-69 | 75/30 | Myzus persicae | soybean |
| | | Aphis gossypii | corn |
| | | Aphis spp. | cotton |
| | | Rhopalosiphum padi | rice |
| | | Sitobion spp. | vegetables |
| | | Bemisia tabaci | cereals |
| | | Bemisia spp. | pome fruits |
| | | Trialeurodes vaporarium | stone fruits |
| | | Nilaparvata lugens | citrus |
| | | Laodelphax striatellus | |
| | | Sogatella furcifera | |
| | | Spodoptera frugiperda | |
| | | Spodoptera spp. | |
| | | Helicoverpa armigera | |
| | | Heliothis spp. | |
| | | Chrysodeixis includens | |
| | | Scirpophaga incertulas | |
| | | Chilo spp. | |
| | | Plutella xylostella | |
| | | Tuta absoluta | |
| | | Cydia pomonella | |
| | | Leucinodes spp. | |
| | | Euschistus heros | |
| | | Nezara viridula | |
| | | Piezodorus spp. | |
| | | Dichelops spp. | |
| | | Frankliniella occidentalis | |
| | | Thrips tabaci | |
| | | Thrips palmi | |
| | | Thrips spp. | |
| | | Scirtothrips dorsalis | |
| | | Diabrotica virgifera | |
| | | virgifera | |
| | | Diabrotica spp. | |
| | | Agriotes spp. | |
| | | Limonius spp. | |
| | | Melanotus spp. | |
| | | wireworms | |
| A-1/B-70 | 75/30 | Myzus persicae | soybean |
| | | Aphis gossypii | corn |
| | | Aphis spp. | cotton |
| | | Rhopalosiphum padi | rice |
| | | Sitobion spp. | vegetables |
| | | Bemisia tabaci | cereals |
| | | Bemisia spp. | pome fruits |
| | | Trialeurodes vaporarium | stone fruits |
| | | Nilaparvata lugens | citrus |
| | | Laodelphax striatellus | |
| | | Sogatella furcifera | |
| | | Spodoptera frugiperda | |
| | | Spodoptera spp. | |
| | | Helicoverpa armigera | |
| | | Heliothis spp. | |
| | | Chrysodeixis includens | |
| | | Scirpophaga incertulas | |
| | | Chilo spp. | |
| | | Plutella xylostella | |
| | | Tuta absoluta | |
| | | Cydia pomonella | |
| | | Leucinodes spp. | |
| | | Euschistus heros | |
| | | Nezara viridula | |
| | | Piezodorus spp. | |
| | | Dichelops spp. | |
| | | Frankliniella occidentalis | |
| | | Thrips tabaci | |
| | | Thrips palmi | |
| | | Thrips spp. | |
| | | Scirtothrips dorsalis | |
| | | Diabrotica virgifera | |
| | | virgifera | |
| | | Diabrotica spp. | |
| | | Agriotes spp. | |
| | | Limonius spp. | |
| | | Melanotus spp. | |
| | | wireworms | |
| A-1/B-71 | 75/30 | Myzus persicae | soybean |
| | | Aphis gossypii | corn |
| | | Aphis spp. | cotton |
| | | Rhopalosiphum padi | rice |
| | | Sitobion spp. | vegetables |
| | | Bemisia tabaci | cereals |
| | | Bemisia spp. | pome fruits |
| | | Trialeurodes vaporarium | stone fruits |
| | | Nilaparvata lugens | citrus |
| | | Laodelphax striatellus | |
| | | Sogatella furcifera | |
| | | Spodoptera frugiperda | |
| | | Spodoptera spp. | |
| | | Helicoverpa armigera | |
| | | Heliothis spp. | |
| | | Chrysodeixis includens | |
| | | Scirpophaga incertulas | |
| | | Chilo spp. | |
| | | Plutella xylostella | |
| | | Tuta absoluta | |
| | | Cydia pomonella | |
| | | Leucinodes spp. | |
| | | Euschistus heros | |
| | | Nezara viridula | |
| | | Piezodorus spp. | |
| | | Dichelops spp. | |
| | | Frankliniella occidentalis | |
| | | Thrips tabaci | |
| | | Thrips palmi | |
| | | Thrips spp. | |
| | | Scirtothrips dorsalis | |
| | | Diabrotica virgifera | |
| | | virgifera | |
| | | Diabrotica spp. | |
| | | Agriotes spp. | |
| | | Limonius spp. | |
| | | Melanotus spp. | |
| | | wireworms | |
| A-1/B-140 | 75/50 | Myzus persicae | soybean |
| | | Aphis gossypii | corn |
| | | Aphis spp. | cotton |
| | | Rhopalosiphum padi | rice |
| | | Sitobion spp. | vegetables |
| | | Bemisia tabaci | cereals |
| | | Bemisia spp. | pome fruits |
| | | Trialeurodes vaporarium | stone fruits |
| | | Nilaparvata lugens | citrus |
| | | Laodelphax striatellus | |
| | | Sogatella furcifera | |
| | | Spodoptera frugiperda | |
| | | Spodoptera spp. | |
| | | Helicoverpa armigera | |
| | | Heliothis spp. | |
| | | Chrysodeixis includens | |
| | | Scirpophaga incertulas | |
| | | Chilo spp. | |
| | | Plutella xylostella | |
| | | Tuta absoluta | |
| | | Cydia pomonella | |
| | | Leucinodes spp. | |
| | | Euschistus heros | |
| | | Nezara viridula | |
| | | Piezodorus spp. | |
| | | Dichelops spp. | |
| | | Frankliniella occidentalis | |
| | | Thrips tabaci | |
| | | Thrips palmi | |
| | | Thrips spp. | |
| | | Scirtothrips dorsalis | |
| | | Diabrotica virgifera | |
| | | virgifera | |
| | | Diabrotica spp. | |
| | | Agriotes spp. | |
| | | Limonius spp. | |
| | | Melanotus spp. | |
| | | wireworms | |
| A-1/B-141 | 75/100 | Myzus persicae | soybean |
| | | Aphis gossypii | corn |
| | | Aphis spp. | cotton |
| | | Rhopalosiphum padi | rice |
| | | Sitobion spp. | vegetables |
| | | Bemisia tabaci | cereals |
| | | Bemisia spp. | pome fruits |
| | | Trialeurodes vaporarium | stone fruits |
| | | Nilaparvata lugens | citrus |
| | | Laodelphax striatellus | |
| | | Sogatella furcifera | |
| | | Spodoptera frugiperda | |
| | | Spodoptera spp. | |
| | | Helicoverpa armigera | |
| | | Heliothis spp. | |
| | | Chrysodeixis includens | |
| | | Scirpophaga incertulas | |
| | | Chilo spp. | |
| | | Plutella xylostella | |
| | | Tuta absoluta | |
| | | Cydia pomonella | |
| | | Leucinodes spp. | |
| | | Euschistus heros | |
| | | Nezara viridula | |
| | | Piezodorus spp. | |
| | | Dichelops spp. | |
| | | Frankliniella occidentalis | |
| | | Thrips tabaci | |
| | | Thrips palmi | |
| | | Thrips spp. | |
| | | Scirtothrips dorsalis | |
| | | Diabrotica virgifera | |
| | | virgifera | |
| | | Diabrotica spp. | |
| | | Agriotes spp. | |
| | | Limonius spp. | |
| | | Melanotus spp. | |
| | | wireworms | |
| A-1/B-142 | 75/150 | Myzus persicae | soybean |
| | | Aphis gossypii | corn |
| | | Aphis spp. | cotton |
| | | Rhopalosiphum padi | rice |
| | | Sitobion spp. | vegetables |
| | | Bemisia tabaci | cereals |
| | | Bemisia spp. | canola |
| | | Trialeurodes vaporarium | sugarcane |
| | | Nilaparvata lugens | sugarbeet |
| | | Laodelphax striatellus | potatoes |
| | | Sogatella furcifera | |
| | | Euschistus heros | |
| | | Nezara viridula | |
| | | Piezodorus spp. | |
| | | Dichelops spp. | |
| | | Frankliniella occidentalis | |
| | | Thrips tabaci | |
| | | Thrips palmi | |
| | | Thrips spp. | |
| | | Scirtothrips dorsalis | |
| | | Pratylenchus spp. | |
| | | Meloidogyne spp. | |
| | | Heterodera spp. | |
| A-1/B-143 | 75 75 | Myzus persicae | soybean |
| | | Aphis gossypii | cotton |
| | | Aphis spp. | vegetables |
| | | Rhopalosiphum padi | pome fruits |
| | | Sitobion spp. | stone fruits |
| | | Bemisia tabaci | citrus |
| | | Bemisia spp. | cereals |
| | | Trialeurodes vaporarium | rice |
| | | Nilaparvata lugens | |
| | | Laodelphax striatellus | |
| | | Sogatella furcifera | |
| A-1/B-144 | 75 300 | Myzus persicae | soybean |
| | | Aphis gossypii | corn |
| | | Aphis spp. | cotton |
| | | Rhopalosiphum padi | rice |
| | | Sitobion spp. | vegetables |
| | | Bemisia tabaci | cereals |
| | | Bemisia spp. | pome fruits |
| | | Trialeurodes vaporarium | stone fruits |
| | | Nilaparvata lugens | citrus |
| | | Laodelphax striatellus | sugarcane |
| | | Sogatella furcifera | |
| | | Spodoptera frugiperda | |
| | | Spodoptera spp. | |
| | | Helicoverpa armigera | |
| | | Heliothis spp. | |
| | | Chrysodeixis includens | |
| | | Scirpophaga incertulas | |
| | | Chilo spp. | |
| | | Plutella xylostella | |
| | | Tuta absoluta | |
| | | Cydia pomonella | |
| | | Leucinodes spp. | |
| | | Euschistus heros | |
| | | Nezara viridula | |
| | | Piezodorus spp. | |
| | | Dichelops spp. | |
| | | Frankliniella occidentalis | |
| | | Thrips tabaci | |
| | | Thrips palmi | |
| | | Thrips spp. | |
| | | Scirtothrips dorsalis | |
| | | Diabrotica virgifera | |
| | | virgifera | |
| | | Diabrotica spp. | |
| | | Agriotes spp. | |
| | | Limonius spp. | |
| | | Melanotus spp. | |
| | | wireworms | |
| | | Diatraea spp. | |
| | | Elasmopalpus spp. | |

In a further aspect of the invention, a combination is made available comprising, as component (A), a compound of formula A-1 and as component (B), one or more selected from B-1 to B-12, B-31, B-32, B-38, B-40, B-42, B-44, B-46, B-51, B-54, B-55, B-60, B-61, B-62, B-63, B-66 B-68, B-69, B-70, B-71, B-72 to B-92, B-141 and B-142. Also made available is a method of controlling or preventing damage to a plant, which comprises applying, in any desired sequence or simultaneously, on the plant, the locus thereof or its propagation material, the combination defined in this paragraph.

In a further aspect of the invention, a combination is made available comprising, as component (A), a compound of formula A-2 and as component (B), one or more selected from B-1 to B-12, B-31, B-32, B-38, B-40, B-42, B-44, B-46, B-51, B-54, B-55, B-60, B-61, B-62, B-63, B-66, B-68, B-69, B-70, B-71, B-72 to B-92, B-141 and B-142. Also made available is a method of controlling or preventing damage to a plant, which comprises applying, in any desired sequence or simultaneously, on the plant, the locus thereof or its propagation material, the combination defined in this paragraph.

In a further aspect of the invention, a combination is made available comprising, as component (A), a compound of formula A-3 and as component (B), one or more selected from B-1 to B-12, B-31, B-32, B-38, B-40, B-42, B-44, B-46, B-51, B-54, B-55, B-60, B-61, B-62, B-63, B-66 B-68, B-69, B-70, B-71, B-72 to B-92, B-141 and B-142. Also made available is a method of controlling or preventing damage to a plant, which comprises applying, in any desired sequence or simultaneously, on the plant, the locus thereof or its propagation material, the combination defined in this paragraph.

In a further aspect of the invention, a combination is made available comprising, as component (A), a compound of formula A-4 and as component (B), one or more selected from B-1 to B-12, B-31, B-32, B-38, B-40, B-42, B-44, B-46, B-51, B-54, B-55, B-60, B-61, B-62, B-63, B-66 B-68, B-69, B-70, B-71, B-72 to B-92, B-141 and B-142. Also made available is a method of controlling or preventing damage to a plant, which comprises applying, in any desired sequence or simultaneously, on the plant, the locus thereof or its propagation material, the combination defined in this paragraph.

In a further aspect of the invention, a combination is made available comprising, as component (A), a compound of formula A-5 and as component (B), one or more selected from B-1 to B-12, B-31, B-32, B-38, B-40, B-42, B-44, B-46, B-51, B-54, B-55, B-60, B-61, B-62, B-63, B-66 B-68, B-69, B-70, B-71, B-72 to B-92, B-141 and B-142. Also made available is a method of controlling or preventing damage to a plant, which comprises applying, in any desired sequence or simultaneously, on the plant, the locus thereof or its propagation material, the combination defined in this paragraph.

In a further aspect of the invention, a combination is made available comprising, as component (A), a compound of formula A-1 and as component (B), one or more selected from B-43, B-52, B-53, B-58, B-65, and B-140, wherein the ratio by weight between compound A-1 and a compound of component (B) is 500:1 to 1:500, 100:1 to 1:100, 50:1 to 1:50, or 20:1 to 1:20, 10:1 to 1:10; 5:1 to 1:5; or 3.1 to 1:3 by weight. Also made available is a method of controlling or preventing damage to a plant, which comprises applying, in any desired sequence or simultaneously, on the plant, the locus thereof or its propagation material, the combination defined in this paragraph.

In a further aspect of the invention, a combination is made available comprising, as component (A), a compound of formula A-2 and as component (B), one or more selected from B-43, B-52, B-53, B-58, B-65, and B-140, wherein the ratio by weight between compound A-1 and a compound of component (B) is 500:1 to 1:500, 100:1 to 1:100, 50:1 to 1:50, or 20:1 to 1:20, 10:1 to 1:10; 5:1 to 1:5; or 3.1 to 1:3 by weight. Also made available is a method of controlling or preventing damage to a plant, which comprises applying, in any desired sequence or simultaneously, on the plant, the locus thereof or its propagation material, the combination defined in this paragraph.

In a further aspect of the invention, a combination is made available comprising, as component (A), a compound of formula A-3 and as component (B), one or more selected from B-43, B-52, B-53, B-58, B-65, and B-140, wherein the ratio by weight between compound A-1 and a compound of component (B) is 500:1 to 1:500, 100:1 to 1:100, 50:1 to 1:50, or 20:1 to 1:20, 10:1 to 1:10; 5:1 to 1:5; or 3.1 to 1:3 by weight. Also made available is a method of controlling or preventing damage to a plant, which comprises applying, in any desired sequence or simultaneously, on the plant, the locus thereof or its propagation material, the combination defined in this paragraph.

In a further aspect of the invention, a combination is made available comprising, as component (A), a compound of formula A-4 and as component (B), one or more selected from B-43, B-52, B-53, B-58, B-65, and B-140, wherein the ratio by weight between compound A-1 and a compound of component (B) is 500:1 to 1:500, 100:1 to 1:100, 50:1 to 1:50, or 20:1 to 1:20, 10:1 to 1:10; 5:1 to 1:5; or 3.1 to 1:3 by weight. Also made available is a method of controlling or preventing damage to a plant, which comprises applying, in any desired sequence or simultaneously, on the plant, the locus thereof or its propagation material, the combination defined in this paragraph.

In a further aspect of the invention, a combination is made available comprising, as component (A), a compound of formula A-5 and as component (B), one or more selected from B-43, B-52, B-53, B-58, B-65, and B-140, wherein the ratio by weight between compound A-1 and a compound of component (B) is 500:1 to 1:500, 100:1 to 1:100, 50:1 to 1:50, or 20:1 to 1:20, 10:1 to 1:10; 5:1 to 1:5; or 3.1 to 1:3 by weight. Also made available is a method of controlling or preventing damage to a plant, which comprises applying, in any desired sequence or simultaneously, on the plant, the locus thereof or its propagation material, the combination defined in this paragraph.

Table 3 sets out certain combinations for controlling or preventing infestation of animals. Table 3 also lists key pests against which the combinations are particularly effective and key crops for which the inventive combinations are particularly advantageous. Each combination of components (A) and (B) and each combination of animal and pest are individually exemplified by Table 3.

TABLE 3

| Combination | | Animal | Pest |
|---|---|---|---|
| A-1 | B-33 | cat, dog, beef cattle, | ticks, fleas, lice, flies, mosquitos; |
| | | sheep, goat and pig | nematodes, cestodes, hookworms, |
| | | | roundworms |
| A-1 | B-34 | cat, dog, beef cattle, | ticks, fleas, lice, flies, mosquitos; |
| | | sheep, goat and pig | nematodes, cestodes, hookworms, |
| | | | roundworms |
| A-1 | B-35 | cat, dog, beef cattle, | ticks, fleas, lice, flies, mosquitos; |
| | | sheep, goat and pig | nematodes, cestodes, hookworms, |
| | | | roundworms |
| A-1 | B-36 | cat, dog, beef cattle, | ticks, fleas, lice, flies, mosquitos; |
| | | sheep, goat and pig | nematodes, cestodes, hookworms, |
| | | | roundworms |
| A-1 | B-37 | cat, dog, beef cattle, | ticks, fleas, lice, flies, mosquitos; |
| | | sheep, goat and pig | nematodes, cestodes, hookworms, |
| | | | roundworms |

### BIOLOGICAL EXAMPLES

The synergistic biological activity of combinations of component (A) and component (B) as disclosed herein was assessed using a Myzus persicae (Green peach aphid) feeding assay. Test compounds prepared from 10′000 ppm DMSO stock solutions were applied by pipette into 24-well microtiter plates and mixed with sucrose solution. The plates were closed with a stretched Parafilm. A plastic stencil with 24 holes was placed onto the plate and pea seedlings infested with a Myzus persicae population of mixed ages were placed directly on the Parafilm. The infested plate was closed with a gel blotting paper and another plastic stencil and then turned upside down. The samples were assessed for mortality 5 days after infestation.

| Component A | Component B | | |
|---|---|---|---|
| A-1 | B-3 | Mortality (%) | |
| [AI] (mg/L) | [AI] (mg/L) | Expected (%) | Observed (%) |
| 0.375 | 0.312 | 75 | 75 |
| 0.188 | 0.156 | 75 | 62 |
| 0.094 | 0.078 | 50 | 37 |
| 0.047 | 0.039 | 25 | 0 |
| 0.188 | 0.625 | 62 | 75 |
| 0.094 | 0.312 | 37 | 62 |
| 0.047 | 0.156 | 0 | 37 |
| 0.094 | 1.25 | 37 | 50 |
| 0.047 | 0.625 | 0 | 25 |
| 0.023 | 0.312 | 0 | 0 |
| Component A | Component B | | |
| A-1 | B-11 | Mortality (%) | |
| [AI] (mg/L) | [AI] (mg/L) | Expected (%) | Observed (%) |
| 0.375 | 0.047 | 75 | 75 |
| 0.188 | 0.023 | 62 | 75 |
| 0.094 | 0.012 | 35 | 37 |
| 0.047 | 0.006 | 0 | 25 |
| 0.188 | 0.094 | 62 | 75 |
| 0.094 | 0.047 | 35 | 37 |
| 0.047 | 0.023 | 0 | 12 |
| 0.023 | 0.012 | 0 | 12 |
| 0.094 | 0.188 | 35 | 62 |
| 0.047 | 0.094 | 0 | 25 |
| 0.023 | 0.047 | 0 | 0 |
| 0.012 | 0.023 | 0 | 0 |
| 0.047 | 0.375 | 75 | 75 |
| 0.023 | 0.188 | 0 | 25 |
| 0.012 | 0.094 | 0 | 0 |
| 0.006 | 0.047 | 0 | 0 |
| Component A | Component B | | |
| A-1 | B-46 | Mortality (%) | |
| [AI] (mg/L) | [AI] (mg/L) | Expected (%) | Observed (%) |
| 0.375 | 0.013 | 75 | 75 |
| 0.188 | 0.006 | 62 | 75 |
| 0.094 | 0.003 | 35 | 37 |
| 0.047 | 0.002 | 0 | 12 |
| 0.094 | 0.05 | 42 | 75 |
| 0.047 | 0.025 | 0 | 12 |
| 0.023 | 0.013 | 0 | 0 |
| 0.094 | 0.2 | 100 | 100 |
| 0.047 | 0.1 | 62 | 75 |
| 0.023 | 0.05 | 12 | 37 |
| Component A | Component B | | |
| A-1 | B-54 | Mortality (%) | |
| [AI] (mg/L) | [AI] (mg/L) | Expected (%) | Observed (%) |
| 0.375 | 0.006 | 75 | 75 |
| 0.188 | 0.003 | 62 | 75 |
| 0.094 | 0.002 | 37 | 50 |
| 0.047 | 0.001 | 0 | 25 |
| 0.188 | 0.013 | 62 | 75 |
| 0.094 | 0.006 | 37 | 50 |
| 0.047 | 0.003 | 0 | 12 |
| 0.023 | 0.002 | 0 | 0 |
| 0.094 | 0.025 | 84 | 75 |
| 0.047 | 0.013 | 0 | 12 |
| 0.023 | 0.006 | 0 | 0 |
| Component A | Component B | | |
| A-1 | B-64 | Mortality (%) | |
| [AI] (mg/L) | [AI] (mg/L) | Expected (%) | Observed (%) |
| 0.188 | 0.312 | 62 | 62 |
| 0.094 | 0.156 | 35 | 62 |
| 0.047 | 0.078 | 0 | 37 |
| 0.188 | 1.25 | 62 | 75 |
| 0.094 | 0.625 | 35 | 50 |
| 0.047 | 0.312 | 0 | 0 |
| 0.188 | 5 | 62 | 75 |
| 0.094 | 2.5 | 35 | 75 |
| 0.047 | 1.25 | 0 | 37 |
| 0.023 | 0.625 | 0 | 0 |
| 0.094 | 10 | 59 | 75 |
| 0.047 | 5 | 0 | 62 |
| 0.023 | 2.5 | 0 | 37 |
| 0.012 | 1.25 | 0 | 0 |
| Component A | Component B | | |
| A-1 | B-71 | Mortality (%) | |
| [AI] (mg/L) | [AI] (mg/L) | Expected (%) | Observed (%) |
| 0.375 | 2.5 | 75 | 75 |
| 0.188 | 1.25 | 62 | 75 |
| 0.094 | 0.625 | 35 | 37 |
| 0.047 | 0.312 | 0 | 25 |
| 0.188 | 5 | 62 | 75 |
| 0.094 | 2.5 | 35 | 62 |
| 0.047 | 1.25 | 0 | 25 |
| 0.023 | 0.625 | 0 | 0 |
| 0.094 | 10 | 83 | 75 |
| 0.047 | 5 | 0 | 75 |
| 0.023 | 2.5 | 0 | 37 |
| 0.012 | 1.25 | 0 | 0 |

1. A composition comprising, as component (A), a compound of formula (I): 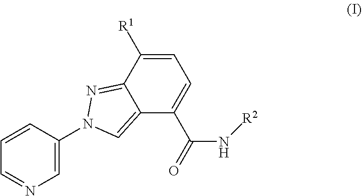
wherein:
R¹ is hydrogen or fluorine; and
R² is: 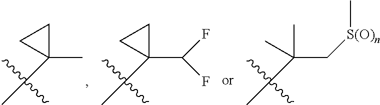
wherein n is 0, 1 or 2;
or a salt thereof;
and, as component (B), at least one compound selected from the group consisting of:
B-1 (S)-N-[[2-fluoro-4-[2-hydroxy-3-(3,4,5-trichlorophenyl)-3-(trifluoromethyl)pyrrolidin-1-yl]phenyl]methyl]cyclopropanecarboxamide (CAS 2044701-44-0)
B-2 N-[[2-fluoro-4-[2-hydroxy-3-(3,4,5-trichlorophenyl)-3-(trifluoromethyl)pyrrolidin-1-yl]phenyl]methyl]cyclopropanecarboxamide (CAS 1365070-72-9)
B-3 spidoxamat (CAS 907187-07-9)
B-4 fenmezoditiaz (CAS 2413390-32-4)
B-5 3-(2-chlorothiazol-5-yl)-8-methyl-5-oxo-6-phenyl-2,3-dihydrothiazolo[3,2-a]pyrimidin-8-ium-7-olate (CAS 2413839-57-1)
B-6 (3R)-3-(2-chlorothiazol-5-yl)-8-methyl-5-oxo-6-phenyl-2,3-dihydrothiazolo[3,2-a]pyrimidin-8-ium-7-olate, enolate tautomer (CAS 2413839-58-2)
B-7 (3R)-3-(2-chlorothiazol-5-yl)-8-methyl-5-oxo-6-phenyl-2,3-dihydrothiazolo[3,2-a]pyrimidin-8-ium-7-olate, diketone (CAS 2246757-58-2)
B-8 1-[6-(2,2-difluoro-7-methyl-[1,3]dioxolo[4,5-f]benzimidazol-6-yl)-5-ethylsulfonyl-3-pyridyl]cyclopropanecarbonitrile (CAS 2408220-94-8)
B-9 2-[3-ethylsulfonyl-5-(trifluoromethyl)pyrazolo[1,5-a]pyridin-2-yl]-3-methyl-6-(trifluoromethyl)imidazo[4,5-b]pyridine (CAS 2133042-44-9)
B-10 2-[3-ethylsulfonyl-6-(trifluoromethyl)pyrazolo[1,5-a]pyridin-2-yl]-3-methyl-6-(trifluoromethyl)imidazo[4,5-b]pyridine (CAS 2133042-31-4)
B-11 (3-ethylsulfonyl-2-pyridyl)-5-(2,2,3,3,3-pentafluoropropoxy)pyrazine (CAS 2095470-94-1)
B-12 2-[3-ethylsulfonyl-6-(1,2,4-triazol-1-yl)-2-pyridyl]-3-methyl-6-(trifluoromethyl)imidazo[4,5-b]pyridine (CAS 1808115-49-2)
B-13 carbosulfan (CAS 55285-14-8)
B-14 1-(2-cyanoethyl)-4-oxo-3-[3-(trifluoromethyl)phenyl]pyrido[1,2-a]pyrimidin-1-ium-2-olate (CAS 2032403-97-5)
B-15 2-(5-cyclopropyl-3-ethylsulfonyl-2-pyridyl)-5-(trifluoromethylsulfonyl)-1,3-benzoxazole (CAS 2128706-05-6)
B-16 6-(5-cyclopropyl-3-ethylsulfonyl-2-pyridyl)-2,2-difluoro-7-methyl-[1,3]dioxolo[4,5-f]benzimidazole (CAS 2408220-91-5)
B-17 azocyclotin (CAS 41083-11-8)
B-18 pyrimidifen (CAS 105779-78-0)
B-19 fluacrypyrim (CAS 229977-93-9)
B-20 diflovidazin (CAS 162320-67-4)
B-21 (R)-(2Z)-2-[2-fluoro-4-methyl-5-(2,2,2-trifluoroethylsulfinyl)phenyl]imino-3-(2,2,2-trifluoroethyl)thiazolidin-4-one (CAS 2377084-09-6)
B-22 2-[2-fluoro-4-methyl-5-(2,2,2-trifluoroethylsulfinyl)phenyl]imino-3-(2,2,2-trifluoroethyl)thiazolidin-4-one (CAS 1445684-82-1)
B-23 (2Z)-2-[2-fluoro-4-methyl-5-(2,2,2-trifluoroethylsulfinyl)phenyl]imino-3-(2,2,2-trifluoroethyl)thiazolidin-4-one (CAS 1445683-71-5)
B-24 4-dimethyl-2-[2-(3-pyridyl)indazol-5-yl]-1,2,4-triazolidine-3,5-dione (CAS 2171099-09-3)
B-25 N-[3-chloro-1-(3-pyridyl)pyrazol-4-yl]-2-methylsulfonyl-propanamide (CAS 2396747-83-2)
B-26 lepimectin (CAS 863549-51-3)
B-27 milbemectin (CAS 1799297-76-9 and CAS 51596-10-2)
B-28 cyproflanilide (CAS 2375110-88-4)
B-29 thiocyclam (CAS 31895-21-3)
B-30 guadipyr (CAS 1227849-60-6)
B-31 5-[[(1R,3R)-3-[3,5-bis(trifluoromethyl)phenyl]-2,2-dichloro-cyclopropanecarbonyl]amino]-2-chloro-N-[3-[(2,2-difluoroacetyl)amino]-2,4-difluoro-phenyl]benzamide) (CAS 2220132-55-6)
B-32 5-[[trans-3-[3,5-bis(trifluoromethyl)phenyl]-2,2-dichloro-cyclopropanecarbonyl]amino]-2-chloro-N-[3-[(2,2-difluoroacetyl)amino]-2,4-difluoro-phenyl]benzamide
B-33 afoxolaner (CAS 1093861-60-9)
B-34 fluralaner (CAS 864731-61-3)
B-35 sarolaner (CAS 1398609-39-6)
B-36 ivermectin (CAS 70288-86-7)
B-37 tigolaner (CAS 1621436-41-6)
B-72 oxathiapiprolin
B-73 iprodione
B-74 fludioxonil
B-75 mefenoxam or metalaxyl-M
B-76 metalaxyl
B-77 penflufen
B-78 fluindapyr
B-79 penthiopyrad
B-80 furametpyr
B-81 pydiflumetofen
B-82 sedaxane
B-83 thiabendazole
B-84 mefentrifluconazole
B-85 prothioconazole
B-86 triticonazole
B-87 flutriafol
B-88 difenconazole
B-89 ipconazole
B-90 azoxystrobin
B-91 fluoxastrobin
B-92 picarbutrazox
B-93 amisulbrom
B-94 cyazofamid
B-95 hymexazole
B-96 fluopicolide
B-97 fluoxapiprolin
B-98 cyprodinil
B-99 thiram
B-100 florylpicoxamid
B-101 fenamidone
B-102 famoxadone
B-103 oxadixyl
B-104 acibenzolar-S-methyl
B-105 boscalid
B-106 bixafen
B-107 fluopyram
B-108 fluxapyroxad
B-109 isoflucypram
B-110 flutolanil
B-111 pyraziflumid
B-112 pyrapropoyne
B-113 inpyrfluxam
B-114 carboxin
B-115 zoxamide
B-116 thiophanate-methyl
B-117 ethaboxam
B-118 dimethomorph
B-119 metconazole
B-120 prochloraz
B-121 tebuconazole
B-122 triadimenol
B-123 fluquinconazole
B-124 myclobutanil
B-125 imazalil
B-126 oxpoconazole
B-127 cyproconazole
B-128 pyraclostrobin
B-129 picoxystrobin
B-130 enestrobin
B-131 fenaminostrobin
B-132 mandestrobin
B-133 metyltetraprole
B-134 fluazinam
B-135 quinofumelin
B-136 cyflufenamid (CAS 180409-60-3)
B-137 ipflufenoquin
B-138 cymoxanil
and
B-139 copper
or salts thereof.

2. A composition according to claim 1 wherein the ratio by weight of (A) to (B) is from 500:1 to 1:500, 100:1 to 1:100, 50:1 to 1:50 or 20:1 to 1:20, or preferably 10:1 to 1:10 or 5:1 to 1:5; more preferably 3.1 to 1:3.

3. A composition comprising, as component (A), a compound of formula (I): 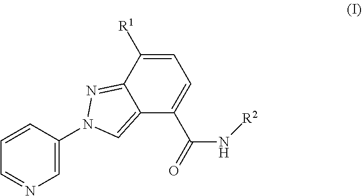
wherein:
R¹ is hydrogen or fluorine; and
R² is: 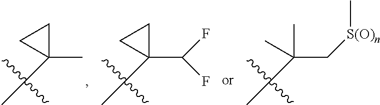
wherein n is 0, 1 or 2;
or a salt thereof;
and, as component (B), at least one compound selected from the group consisting of:
B-38 afidopyropen (CAS 915972-17-7)
B-39 flonicamid (CAS 158062-67-0)
B-40 fluxametamide (CAS 928783-29-3)
B-41 spirotetramat (CAS 203313-25-1)
B-42 triflumezopyrim (CAS 1263133-33-0)
B-43 broflanilide (CAS 1207727-04-5)
B-44 nicofluprole (CAS 1771741-86-6)
B-45 spinetoram (CAS 935545-74-7)
B-46 thiacloprid (CAS 111988-49-9)
B-47 flupyradifurone (CAS 951659-40-8)
B-48 imidacloprid (CAS 138261-41-3)
B-49 sulfoxaflor (CAS 946578-00-3)
B-50 acetamiprid (CAS 135410-20-7)
B-51 nitenpyram (CAS 150824-47-8)
B-52 clothianidin (CAS 210880-92-5)
B-53 thiamethoxam (CAS 153719-23-4)
B-54 tetraniliprole (CAS 1229654-66-3)
B-55 flubendiamide (CAS 272451-65-7)
B-56 chlorantraniliprole (CAS 500008-45-7)
B-57 cyantraniliprole (CAS 736994-63-1)
B-58 oxazosulfyl (CAS 1403615-77-9)
B-59 indoxacarb (CAS 173584-44-6)
B-60 cyfluthrin (CAS 68359-37-5)
B-61 deltamethrin (CAS 52918-63-5)
B-62 acrinathrin (CAS 101007-06-1)
B-63 zeta-cypermethrin (CAS 1315501-18-8)
B-64 bifenthrin (CAS 82657-04-3)
B-65 gamma-cyhalothrin (CAS 76703-62-3)
B-66 alpha-cypermethrin (CAS 67375-30-8)
B-67 permethrin (CAS 52645-53-1)
B-68 fenpropathrin (CAS 39515-41-8)
B-69 fenvalerate (CAS 51630-58-1)
B-70 esfenvalerate (CAS 66230-04-4)
B-71 lambda-cyhalothrin (CAS 91465-08-6)
B-140 isocycloseram (CAS 2061933-85-3)
B-141 tefluthrin (CAS 79538-32-2)
B-143 dimpropyridaz (CAS 1403615-77-9)
and
B-144 fipronil (CAS 120068-37-3)
or salts thereof;
wherein the composition comprises (A) and (B) in a ratio by weight of 500:1 to 1:500, 100:1 to 1:100, 50:1 to 1:50, or 20:1 to 1:20, 10:1 to 1:10; 5:1 to 1:5; or 3.1 to 1:3 by weight.

4. A composition according to any one of claims 1 to 3 further comprising one or more auxiliaries and/or diluents.

5. A method of controlling or preventing damage to a plant, which comprises applying, in any desired sequence or simultaneously, on the plant, the locus thereof or its propagation material, comprising, as component (A), a compound of formula (I): 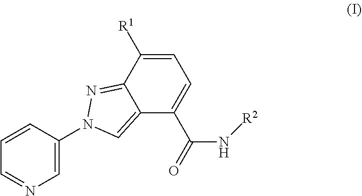
wherein:
R¹ is hydrogen or fluorine; and
R² is: 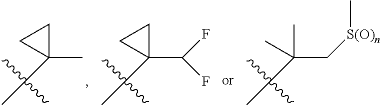
wherein n is 0, 1 or 2;
or an agrochemically acceptable salt thereof; and, as component (B), at least one compound selected from the group consisting of
B-1 (S)-N-[[2-fluoro-4-[2-hydroxy-3-(3,4,5-trichlorophenyl)-3-(trifluoromethyl)pyrrolidin-1-yl]phenyl]methyl]cyclopropanecarboxamide (CAS 2044701-44-0)
B-2 N-[[2-fluoro-4-[2-hydroxy-3-(3,4,5-trichlorophenyl)-3-(trifluoromethyl)pyrrolidin-1-yl]phenyl]methyl]cyclopropanecarboxamide (CAS 1365070-72-9)
B-3 spidoxamat (CAS 907187-07-9)
B-4 fenmezoditiaz (CAS 2413390-32-4)
B-5 3-(2-chlorothiazol-5-yl)-8-methyl-5-oxo-6-phenyl-2,3-dihydrothiazolo[3,2-a]pyrimidin-8-ium-7-olate (CAS 2413839-57-1)
B-6 (3R)-3-(2-chlorothiazol-5-yl)-8-methyl-5-oxo-6-phenyl-2,3-dihydrothiazolo[3,2-a]pyrimidin-8-ium-7-olate, enolate tautomer (CAS 2413839-58-2)
B-7 (3R)-3-(2-chlorothiazol-5-yl)-8-methyl-5-oxo-6-phenyl-2,3-dihydrothiazolo[3,2-a]pyrimidin-8-ium-7-olate, diketone (CAS 2246757-58-2)
B-8 1-[6-(2,2-difluoro-7-methyl-[1,3]dioxolo[4,5-f]benzimidazol-6-yl)-5-ethylsulfonyl-3-pyridyl]cyclopropanecarbonitrile (CAS 2408220-94-8)
B-9 2-[3-ethylsulfonyl-5-(trifluoromethyl)pyrazolo[1,5-a]pyridin-2-yl]-3-methyl-6-(trifluoromethyl)imidazo[4,5-b]pyridine (CAS 2133042-44-9)
B-10 2-[3-ethylsulfonyl-6-(trifluoromethyl)pyrazolo[1,5-a]pyridin-2-yl]-3-methyl-6-(trifluoromethyl)imidazo[4,5-b]pyridine (CAS 2133042-31-4)
B-11 (3-ethylsulfonyl-2-pyridyl)-5-(2,2,3,3,3-pentafluoropropoxy)pyrazine (CAS 2095470-94-1)
B-12 2-[3-ethylsulfonyl-6-(1,2,4-triazol-1-yl)-2-pyridyl]-3-methyl-6-(trifluoromethyl)imidazo[4,5-b]pyridine (CAS 1808115-49-2)
B-13 carbosulfan (CAS 55285-14-8)
B-14 1-(2-cyanoethyl)-4-oxo-3-[3-(trifluoromethyl)phenyl]pyrido[1,2-a]pyrimidin-1-ium-2-olate (CAS 2032403-97-5)
B-15 2-(5-cyclopropyl-3-ethylsulfonyl-2-pyridyl)-5-(trifluoromethylsulfonyl)-1,3-benzoxazole (CAS 2128706-05-6)
B-16 6-(5-cyclopropyl-3-ethylsulfonyl-2-pyridyl)-2,2-difluoro-7-methyl-[1,3]dioxolo[4,5-f]benzimidazole (CAS 2408220-91-5)
B-17 azocyclotin (CAS 41083-11-8)
B-18 pyrimidifen (CAS 105779-78-0)
B-19 fluacrypyrim (CAS 229977-93-9)
B-20 diflovidazin (CAS 162320-67-4)
B-21 (R)-(2Z)-2-[2-fluoro-4-methyl-5-(2,2,2-trifluoroethylsulfinyl)phenyl]imino-3-(2,2,2-trifluoroethyl)thiazolidin-4-one (CAS 2377084-09-6)
B-22 2-[2-fluoro-4-methyl-5-(2,2,2-trifluoroethylsulfinyl)phenyl]imino-3-(2,2,2-trifluoroethyl)thiazolidin-4-one (CAS 1445684-82-1)
B-23 (2Z)-2-[2-fluoro-4-methyl-5-(2,2,2-trifluoroethylsulfinyl)phenyl]imino-3-(2,2,2-trifluoroethyl)thiazolidin-4-one (CAS 1445683-71-5)
B-24 4-dimethyl-2-[2-(3-pyridyl)indazol-5-yl]-1,2,4-triazolidine-3,5-dione (CAS 2171099-09-3)
B-25 N-[3-chloro-1-(3-pyridyl)pyrazol-4-yl]-2-methylsulfonyl-propanamide (CAS 2396747-83-2)
B-26 lepimectin (CAS 863549-51-3)
B-27 milbemectin (CAS 1799297-76-9 and CAS 51596-10-2)
B-28 cyproflanilide (CAS 2375110-88-4)
B-29 thiocyclam (CAS 31895-21-3)
B-30 guadipyr (CAS 1227849-60-6)
B-31 5-[[(1R,3R)-3-[3,5-bis(trifluoromethyl)phenyl]-2,2-dichloro-cyclopropanecarbonyl]amino]-2-chloro-N-[3-[(2,2-difluoroacetyl)amino]-2,4-difluoro-phenyl]benzamide) (CAS 2220132-55-6)
B-32 5-[[trans-3-[3,5-bis(trifluoromethyl)phenyl]-2,2-dichloro-cyclopropanecarbonyl]amino]-2-chloro-N-[3-[(2,2-difluoroacetyl)amino]-2,4-difluoro-phenyl]benzamide
B-33 afoxolaner (CAS 1093861-60-9)
B-34 fluralaner (CAS 864731-61-3)
B-35 sarolaner (CAS 1398609-39-6)
B-36 ivermectin (CAS 70288-86-7)
B-37 tigolaner (CAS 1621436-41-6)
B-72 oxathiapiprolin
B-73 iprodione
B-74 fludioxonil
B-75 mefenoxam or metalaxyl-M
B-76 metalaxyl
B-77 penflufen
B-78 fluindapyr
B-79 penthiopyrad
B-80 furametpyr
B-81 pydiflumetofen
B-82 sedaxane
B-83 thiabendazole
B-84 mefentrifluconazole
B-85 prothioconazole
B-86 triticonazole
B-87 flutriafol
B-88 difenconazole
B-89 ipconazole
B-90 azoxystrobin
B-91 fluoxastrobin
B-92 picarbutrazox
B-93 amisulbrom
B-94 cyazofamid
B-95 hymexazole
B-96 fluopicolide
B-97 fluoxapiprolin
B-98 cyprodinil
B-99 thiram
B-100 florylpicoxamid
B-101 fenamidone
B-102 famoxadone
B-103 oxadixyl
B-104 acibenzolar-S-methyl
B-105 boscalid
B-106 bixafen
B-107 fluopyram
B-108 fluxapyroxad
B-109 isoflucypram
B-110 flutolanil
B-111 pyraziflumid
B-112 pyrapropoyne
B-113 inpyrfluxam
B-114 carboxin
B-115 zoxamide
B-116 thiophanate-methyl
B-117 ethaboxam
B-118 dimethomorph
B-119 metconazole
B-120 prochloraz
B-121 tebuconazole
B-122 triadimenol
B-123 fluquinconazole
B-124 myclobutanil
B-125 imazalil
B-126 oxpoconazole
B-127 cyproconazole
B-128 pyraclostrobin
B-129 picoxystrobin
B-130 enestrobin
B-131 fenaminostrobin
B-132 mandestrobin
B-133 metyltetraprole
B-134 fluazinam
B-135 quinofumelin
B-136 cyflufenamid (CAS 180409-60-3)
B-137 ipflufenoquin
B-138 cymoxanil
and
B-139 copper
or agrochemically acceptable salts thereof.

6. A composition according to claim 1 or claim 2 or a method according to claim 5 wherein component (B) further comprises at least one compound selected from
B-38 afidopyropen (CAS 915972-17-7)
B-39 flonicamid (CAS 158062-67-0)
B-40 fluxametamide (CAS 928783-29-3)
B-41 spirotetramat (CAS 203313-25-1)
B-42 triflumezopyrim (CAS 1263133-33-0)
B-43 broflanilide (CAS 1207727-04-5)
B-44 nicofluprole (CAS 1771741-86-6)
B-45 spinetoram (CAS 935545-74-7)
B-46 thiacloprid (CAS 111988-49-9)
B-47 flupyradifurone (CAS 951659-40-8)
B-48 imidacloprid (CAS 138261-41-3)
B-49 sulfoxaflor (CAS 946578-00-3)
B-50 acetamiprid (CAS 135410-20-7)
B-51 nitenpyram (CAS 150824-47-8)
B-52 clothianidin (CAS 210880-92-5)
B-53 thiamethoxam (CAS 153719-23-4)
B-54 tetraniliprole (CAS 1229654-66-3)
B-55 flubendiamide (CAS 272451-65-7)
B-56 chlorantraniliprole (CAS 500008-45-7)
B-57 cyantraniliprole (CAS 736994-63-1)
B-58 oxazosulfyl (CAS 1403615-77-9)
B-59 indoxacarb (CAS 173584-44-6)
B-60 cyfluthrin (CAS 68359-37-5)
B-61 deltamethrin (CAS 52918-63-5)
B-62 acrinathrin (CAS 101007-06-1)
B-63 zeta-cypermethrin (CAS 1315501-18-8)
B-64 bifenthrin (CAS 82657-04-3)
B-65 gamma-cyhalothrin (CAS 76703-62-3)
B-66 alpha-cypermethrin (CAS 67375-30-8)
B-67 permethrin (CAS 52645-53-1)
B-68 fenpropathrin (CAS 39515-41-8)
B-69 fenvalerate (CAS 51630-58-1)
B-70 esfenvalerate (CAS 66230-04-4)
B-71 lambda-cyhalothrin (CAS 91465-08-6)
B-140 isocycloseram (CAS 2061933-85-3)
B-141 tefluthrin (CAS 79538-32-2)
B-142 cyclobutrifluram (CAS 1644251-74-0)
B-143 dimpropyridaz (CAS 1403615-77-9)
and
B-144 fipronil (CAS 120068-37-3)
or agrochemically acceptable salts thereof.

7. A method of controlling or preventing damage to a plant, which comprises applying, in any desired sequence or simultaneously, on the plant, the locus thereof or its propagation material, comprising, as component (A), a compound of formula (I): 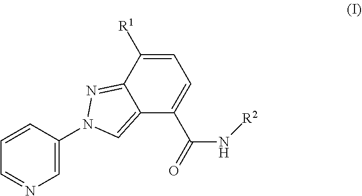
wherein:
R¹ is hydrogen or fluorine; and
R² is: 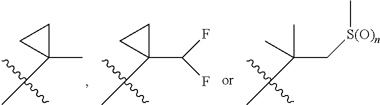
wherein n is 0, 1 or 2;
or an agrochemically acceptable salt thereof; and, as component (B), a compound selected from the group consisting of
B-38 afidopyropen (CAS 915972-17-7)
B-39 flonicamid (CAS 158062-67-0)
B-40 fluxametamide (CAS 928783-29-3)
B-41 spirotetramat (CAS 203313-25-1)
B-42 triflumezopyrim (CAS 1263133-33-0)
B-43 broflanilide (CAS 1207727-04-5)
B-44 nicofluprole (CAS 1771741-86-6)
B-45 spinetoram (CAS 935545-74-7)
B-46 thiacloprid (CAS 111988-49-9)
B-47 flupyradifurone (CAS 951659-40-8)
B-48 imidacloprid (CAS 138261-41-3)
B-49 sulfoxaflor (CAS 946578-00-3)
B-50 acetamiprid (CAS 135410-20-7)
B-51 nitenpyram (CAS 150824-47-8)
B-52 clothianidin (CAS 210880-92-5)
B-53 thiamethoxam (CAS 153719-23-4)
B-54 tetraniliprole (CAS 1229654-66-3)
B-55 flubendiamide (CAS 272451-65-7)
B-56 chlorantraniliprole (CAS 500008-45-7)
B-57 cyantraniliprole (CAS 736994-63-1)
B-58 oxazosulfyl (CAS 1403615-77-9)
B-59 indoxacarb (CAS 173584-44-6)
B-60 cyfluthrin (CAS 68359-37-5)
B-61 deltamethrin (CAS 52918-63-5)
B-62 acrinathrin (CAS 101007-06-1)
B-63 zeta-cypermethrin (CAS 1315501-18-8)
B-64 bifenthrin (CAS 82657-04-3)
B-65 gamma-cyhalothrin (CAS 76703-62-3)
B-66 alpha-cypermethrin (CAS 67375-30-8)
B-67 permethrin (CAS 52645-53-1)
B-68 fenpropathrin (CAS 39515-41-8)
B-69 fenvalerate (CAS 51630-58-1)
B-70 esfenvalerate (CAS 66230-04-4)
B-71 lambda-cyhalothrin (CAS 91465-08-6)
B-140 isocycloseram (CAS 2061933-85-3)
B-141 tefluthrin (CAS 79538-32-2)
B-143 dimpropyridaz (CAS 1403615-77-9)
and
B-144 fipronil (CAS 120068-37-3)
or agrochemically acceptable salts thereof;
wherein the composition comprises component (A) and the or each compound of component (B) in a ratio by weight of 500:1 to 1:500, 100:1 to 1:100, 50:1 to 1:50, or 20:1 to 1:20, 10:1 to 1:10; 5:1 to 1:5; or 3.1 to 1:3 by weight.

8. The method according to any one of claims 5 to 7, wherein the rate of application of component (A) is 1 to 2000 g/ha, especially from 10 to 1000 g/ha, more especially from 20 to 200 g/ha; and the rate of application of the or each compound of component (B) is 1 to 2000 g/ha, especially from 10 to 1000 g/ha, more especially from 20 to 200 g/ha.

9. A method of controlling or preventing infestation of an animal, which comprises applying, in any desired sequence or simultaneously, on the animal a combination comprising, as component (A), a compound of formula (I): 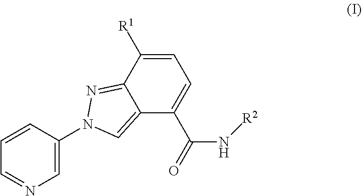
wherein:
R¹ is hydrogen or fluorine; and
R² is: 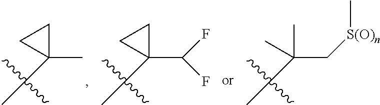
wherein n is 0, 1 or 2;
as defined above or a pharmaceutically or veterinary acceptable salt thereof; and, as component (B), at least one compound selected from the group consisting of
B-33 afoxolaner (CAS 1093861-60-9)
B-34 fluralaner (CAS 864731-61-3)
B-35 sarolaner (CAS 1398609-39-6)
B-36 ivermectin (CAS 70288-86-7)
and
B-37 tigolaner (CAS 1621436-41-6)
or pharmaceutically or veterinary acceptable salts thereof.

10. The use of a combination comprising, as component (A), a compound of formula (I): 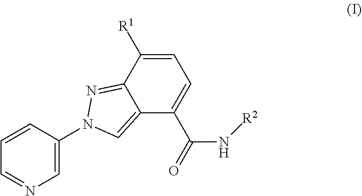
wherein:
R¹ is hydrogen or fluorine; and
R² is: 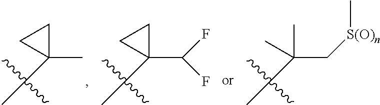
wherein n is 0, 1 or 2;
or a pharmaceutically or veterinary acceptable salt thereof; and, as component (B), at least one compound selected from the group consisting of:
B-33 afoxolaner (CAS 1093861-60-9)
B-34 fluralaner (CAS 864731-61-3)
B-35 sarolaner (CAS 1398609-39-6)
B-36 ivermectin (CAS 70288-86-7)
and
B-37 tigolaner (CAS 1621436-41-6)
or pharmaceutically or veterinary acceptable salts thereof, in controlling or preventing infestation of an animal.

11. The use of a composition as claimed in any one of claims 1 to 4 or claim 6 as an insecticide, acaricide and/or nematicide or for controlling insect, acarina and/or nematode pests.

12. The method according to any one of claims 5 to 9 or the use of claim 10 or claim 11 wherein the combination is applied in succession or simultaneously or as a composition comprising (A) and (B).

13. The method according to any one of claims 5 to 9 or claim 12 or the use according to any one of claims 10 to 12, wherein the combination controls one or more pests selected from insect, acarina, nematode and fungal pests.

14. A composition as claimed in any one of claims 1 to 4 or claim 6, or a method as claimed in any one of claims 5 to 9 or claim 12 or claim 13, or a use as claimed in any one of claims 10 to 13 wherein the compound of formula (I) is a compound of formula (I) in which R¹ is hydrogen and R² is

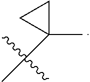 [SMILES: *C1(C)CC1 ; "."]

15. A composition as claimed in any one of claims 1 to 4 or claim 6, or a method as claimed in any one of claims 5 to 9 or claims 12 to 14, or a use as claimed in any one of claims 10 to 14 wherein the compound of formula (I) is a compound of formula (I) in which R¹ is hydrogen and R² is

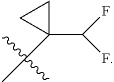 [SMILES: *C1(C(F)F)CC1]

16. A composition as claimed in any one of claims 1 to 4 or claim 6, or a method as claimed in any one of claims 5 to 9 or claims 12 to 15, or a use as claimed in any one of claims 10 to 15 wherein the compound of formula (I) is a compound of formula (I) in which R¹ is fluorine and R² is 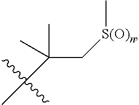
wherein n is 0, 1 or 2.

17. A composition as claimed in any one of claims 1 to 4 or claim 6, or a method as claimed in any one of claims 5 to 9 or claims 12 to 16, or a use as claimed in any one of claims 10 to 16 wherein the compound of formula (I) is

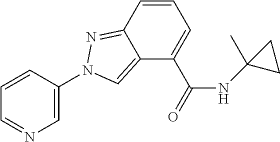 [SMILES: CC1(NC(=O)c2cccc3nn(-c4cccnc4)cc23)CC1]

18. A formulation comprising a composition as claimed in any one of claims 1 to 4, claim 6 or claims 14 to 17, the formulation comprising from 0.01 to 90% by weight of active agents, from 0 to 25% by weight of agriculturally or pharmaceutically acceptable surfactant and from 10 to 99.9 percent by weight of solid or liquid formulation inerts and adjuvants.

19. A concentrate composition for dilution by a user, the concentrate composition comprising a composition as claimed in any one of claims 1 to 4, claim 6 or claims 14 to 17, comprising from 2% to 80% by weight, preferably between about 5% and 70% by weight, of active agents.
